(19) United States
(12) Patent Application Publication
(10) Pub. No.: US 2026/0274978 A1
(43) Pub. Date: Sep. 17, 2026

(54) TARGETING TNFR2 TO TREAT INFECTION AND INFLAMMATION

(71) Applicant: The Johns Hopkins University, Baltimore, MD (US)

(72) Inventors: Nathan Archer, Baltimore, MD (US); Christine Youn, Baltimore, MD (US)

(21) Appl. No.: 19/167,206

(22) PCT Filed: Mar. 27, 2024
(86) PCT No.: PCT/US2024/021691

§ 371 Date: Sep. 19, 2025

Related U.S. Application Data
(60) Provisional application No. 63/493,382, filed on Mar. 31, 2023.

(51) Int. Cl. C07K 16/28; A61P 31/04

(52) U.S. Cl. CPC C07K 16/2878; A61P 31/04; C07K 2317/21; C07K 2317/24

(57) ABSTRACT

Provided herein are methods for treating skin infections and inflammation. TNFR2 agonists and their use in treating bacterial skin infections, such as Staphylococcus aureus also are disclosed.

### STATEMENT OF GOVERNMENTAL INTEREST

This invention was made with government support under AR073665, AR069502, AI146177, and AI111205 awarded by the National Institute of Health. The government has certain rights in the invention.

### BACKGROUND

The skin is a barrier against bacterial infections. Many bacteria come in contact with or reside on the skin but are normally unable to establish an infection. Bacterial skin infections develop when bacteria enter through hair follicles or through small breaks in the skin. Bacterial skin infections can develop after contact with contaminated environments, such as contaminated soil or water. Some infections involve just the skin, and others also involve the soft tissues under the skin.

Many types of bacteria can infect the skin. Staphylococcus aureus is the leading cause of skin and soft tissue infections and is a major health burden due to the emergence of antibiotic-resistant strains. Methicillin-resistant Staphylococcus aureus (also known as MRSA) is a group of common bacteria causing skin infections in the United States. MRSA is resistant to many commonly used antibiotics. With the emergence of antibiotic-resistant S. aureus outpacing the development of novel antibiotics, there is an urgent need to better understand the protective immune mechanisms against S. aureus infections for the development of alternative treatments to existing antibiotics.

### SUMMARY

The presently disclosed subject matter provides drug targets and methods for treating skin infections and inflammation, including but not limited to, S. aureus infections.

In some aspects, the presently disclosed subject matter provides a method for treating a bacterial skin infection in a subject, the method comprising administering to a subject in need of treatment thereof a therapeutically effective amount of a tumor necrosis factor receptor 2 (TNFR2) agonist. The TNFR2 agonist may be an antibody-based therapeutic, such as a monoclonal antibody; a ligand-based TNFR2 agonist; or a small molecule agonist of TNFR2. In some aspects, the TNFR2 agonist is a monoclonal antibody (mAb). In some aspects, the TNFR2 agonist is selected from the group consisting of BI-1910, HFB200301 and MM-401.

In some aspects, the presently disclosed subject matter provides use of a TNFR2 agonist for the treatment of a bacterial skin infection in a subject.

In some aspects, the presently disclosed subject matter provides use of a TNFR2 agonist in the manufacture of a medicament for treatment of a bacterial skin infection in a subject.

The provided uses and methods for treating a bacterial skin infection in a subject can be useful in the treatment of one or more Gram-positive skin pathogens, one or more Gram-negative skin pathogens, or both. In some aspects, the provided uses and methods are useful for treating a bacterial skin infection that is caused by a Gram-positive skin pathogen. In some aspects, the provided uses and methods are useful for treating a bacterial skin infection that is caused by a Gram-negative skin pathogen.

In some aspects, the provided uses and methods are useful for treating bacterial skin infection that is caused by antibiotic-resistant bacteria. In particular aspects, the provided uses and methods are useful for treating a bacterial skin infection that is caused by Staphylococcus aureus (S. aureus), including, but not limited to, a bacterial skin infection that is caused by methicillin-resistant S. aureus. In other aspects, the provided uses and methods are useful for treating a bacterial skin infection that is caused by Pseudomonas aeruginosa (P. aeruginosa).

In some aspects, administering of a therapeutically effective amount of a TNFR2 agonist promotes wound and/or skin lesion healing, reduces the size of one or more skin lesions formed after a bacterial infection, accelerates the rate of reduction in size of one or more skin lesions after a bacterial infection, reduces bacterial burden in a subject and/or at one or more sites of bacterial infection, and/or promotes bacterial clearance after infection.

In certain aspects, a treatment method of the present disclosure (e.g., administering a TNFR2 agonist) results in reducing the size of one or more skin lesions caused by a bacterial infection as compared to the size of the one or more skin lesions prior to treatment.

In certain aspects, a treatment method of the present disclosure (e.g., administering a TNFR2 agonist) results in reducing bacterial burden in the subject receiving treatment (e.g., a TNFR2 agonist) as compared to bacterial burden prior to treatment.

In certain aspects, a treatment method of the present disclosure (e.g., administering a TNFR2 agonist) results in accelerating the rate of reduction in size of one or more skin lesions formed after a bacterial infection as compared to the rate of reduction in size of one or more skin lesions formed after a bacterial infection in a subject not receiving the TNFR2 agonist.

In certain aspects, a treatment method of the present disclosure (e.g., administering a TNFR2 agonist) results in accelerating the rate of bacterial clearance after a bacterial infection as compared to the rate of bacterial clearance after a bacterial infection in a subject not receiving the TNFR2 agonist.

In certain aspects, administering a TNFR2 agonist to a subject in need of treatment for a bacterial infection results in reducing the size of one or more skin lesions formed after the bacterial infection as compared to the size of one or more skin lesions formed after a bacterial infection in a subject not administered the TNFR2 agonist.

In certain aspects, administering a TNFR2 agonist to a subject in need of treatment for a bacterial infection results in reducing bacterial burden in the subject as compared to bacterial burden in a subject in need of treatment for a bacterial infection that is not administered the TNFR2 agonist.

In some aspects, administering of a therapeutically effective amount of a TNFR2 agonist increases neutrophil extracellular trap (NET) formation in a subject receiving the TNFR2 agonist as compared to a subject not administered the TNFR2 agonist. In particular embodiments, the TNFR2 agonist promotes increased NET formation at the infection site. The increased NET formation can be increased suicidal NET formation, increased vital NET formation, or both. In some aspects, administering of a therapeutically effective amount of a TNFR2 agonist does not significantly alter total reactive oxygen species (ROS) production in circulating neutrophils and/or skin neutrophils in a subject receiving the TNFR2 agonist as compared to a subject not receiving the TNFR2 agonist.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

The patent or application file contains at least one drawing executed in color. Copies of this patent or patent application publication with color drawing(s) will be provided by the Office upon request and payment of the necessary fee.

Having thus described the presently disclosed subject matter in general terms, reference will now be made to the accompanying Figures, which are not necessarily drawn to scale, and wherein:

FIG. 1A, FIG. 1B, FIG. 1C, FIG. 1D, FIG. 1E, FIG. 1F, FIG. 1G, FIG. 1H, FIG. 1I, and FIG. 1J demonstrate that TNF from BM-derived immune cells is required for host defense against S. aureus skin infection. S. aureus (3×10⁷ CFU) skin infection was performed on WT and TNF-deficient (TNF^{-/-}) mice (n≥8 per group). (FIG. 1A) Representative photographs of skin lesions. (FIG. 1B) Mean total lesion size (cm²)±SEM. (FIG. 1C) Representative in vivo bioluminescent imaging signals and total flux (photons/s) (log₁₀ scale). (FIG. 1D) Mean total flux (photons/s)±SEM. (FIG. 1E and FIG. 1F) WT or TNF^{-/-} recipient mice were lethally irradiated (8 Gy) and reconstituted with BM from WT or TNF^{-/-} donor mice. After 8 weeks, BM-reconstituted mice were intradermally inoculated with S. aureus (n≥8). (FIG. 1E) Mean total lesion size (cm²) SEM. (FIG. 1F) Mean total flux (photons/s)±SEM. (FIG. 1G) Mean TNF protein levels (picograms per milligram of tissue)±SEM from WT skin biopsies at day 0, 1, 3, and 7 post-infection with S. aureus. (FIG. 1H-FIG. 1J) Infected skin tissue from WT mice was harvested on days 0 through 14 for flow cytometry analysis (n≥8 per group). (FIG. 1H) Total number of neutrophils (PMN), monocytes (Mono), and macrophages (Mac)±SEM on day 0, 1, 3, 7, and 14 post-infection. (FIG. 1I) Total number of TNF+ neutrophils, monocytes, and macrophages±SEM on day 0, 1, 3, 7, and 14 post-infection. (J) Mean TNF+ cells from the CD45+ population (%) SEM on day 3. *P<0.05, †P<0.01, ‡P<0.001, as calculated by a two-way ANOVA test (FIG. 1B, FIG. 1D, FIG. 1E, and FIG. 1F) or a Student's t test (2-tailed) (FIG. 1G). Results are combined from at least two independent experiments:

FIG. 2A, FIG. 2B, FIG. 2C. FIG. 2D, FIG. 2E, FIG. 2F, FIG. 2G, and FIG. 2H demonstrate that TNFR1 and TNFR2 signaling contribute to host defense against S. aureus skin infection. S. aureus (3×10⁷ CFU) skin infection was performed on WT and TNFR1-deficient (TNFR1^{-/-}) and TNFR2-deficient (TNFR2^{-/-}) mice (n≥8 per group). (FIG. 2A) Representative photographs of skin lesions. (FIG. 2B) Mean total lesion size (cm) SEM. (FIG. 2C) Representative in vivo bioluminescent imaging signals and total flux (photons/s) (log₁₀ scale). (FIG. 2D) Mean total flux (photons/s)±SEM. (FIG. 2E-FIG. 2H) WT skin tissue was harvested on day 0 and 3 post-infection for flow cytometry analysis (n≥9). (FIG. 2E and FIG. 2F) Mean TNFR1+ (FIG. 2E) or TNFR2+ (FIG. 2F) cells from live cells (%)±SEM on day 3. (FIG. 2G and FIG. 2H) Total number of TNFR1+(FIG. 2G) or TNFR2+ (FIG. 2H) neutrophils, monocytes, and macrophages±SEM on day 3. Results are combined from at least two independent experiments. *P<0.05, †P<0.01, ‡P<0.001, as calculated by a two-way ANOVA test (FIG. 2B and FIG. 2D) or a Student's t test (2-tailed) (FIG. 2G and FIG. 2H). ns, not significant:

FIG. 3A, FIG. 3B, FIG. 3C, FIG. 3D, FIG. 3E, FIG. 3F, FIG. 3G, and FIG. 3H demonstrate that neutrophil-intrinsic TNFR1 and TNFR2 signaling provide protection against S. aureus skin infections. Neutrophils from TNFR1^{-/-} or WT donor mice were adoptively transferred into (FIG. 3A-FIG. 3D) TNFR1^{-/-} mice or (FIG. 3E-FIG. 3H) neutrophils from TNFR2^{-/-} or WT donor mice into TNFR2^{-/-} mice (n≥10 per group). After 2 hours, the recipient mice were infected intradermally with S. aureus (3×10⁷ CFU) (FIG. 3A and FIG. 3E) Representative photographs of skin lesions. (FIG. 3C and FIG. 3G) Representative in vivo bioluminescent imaging signals and total flux (photons/s) (dogi scale). (FIG. 3B and FIG. 3F) Mean total lesion size (cm²)±SEM. (FIG. 3D and FIG. 3H) Mean total flux (photons/s)±SEM. Results are combined from two independent experiments. *P<0.05, †P<0.01, ‡P<0.001, as calculated by a two-way ANOVA test (FIG. 3B, FIG. 3D. FIG. 3F, and FIG. 3H);

FIG. 4A, FIG. 4B, FIG. 4C, FIG. 4D, and FIG. 4E demonstrate that TNFR1 promotes neutrophilic abscess formation and recruitment during S. aureus skin infections. S. aureus (3×10⁷ CFU) skin infections were performed on WT, TNF^{-/-}, TNFR1^{-/-}, and TNFR2^{-/-} mice and the infected skin tissue was harvested on day 3 post-infection for histology (n≥8 per group) and for flow cytometry analysis (n≥8 per group). (FIG. 4A) Representative H&E skin sections (Scale bars, 500 μm). Black arrows indicate the region of the horizontal area of neutrophilic abscess. (FIG. 4B) Mean neutrophilic abscess area (cm²)±SEM. (FIG. 4C) Representative Gram stain skin sections (Scale bars, 500 μm). (FIG. 4D) Mean bacterial band width (mm)±SEM. (FIG. 4E) Total number of neutrophils, monocytes, and macrophages±SEM from S. aureus-infected skin on day 3 post-infection. Results are combined from at least two independent experiments. †P<0.01, ‡P<0.001, as calculated by a one-way ANOVA multiple comparisons test. ns, not significant;

FIG. 5A, FIG. 5B, FIG. 5C, FIG. 5D, FIG. 5E, FIG. 5F, FIG. 5G, FIG. 5H, and FIG. 5I show that NETs contribute to host defense against S. aureus skin infection. S. aureus (3×10⁷ CFU) skin infection was performed on WT and PAD4-deficient (PAD4^{-/-}) mice (n≥8 per group). (FIG. 5A) Representative photographs of skin lesions. (FIG. 5B) Mean total lesion size (cm²)±SEM. (FIG. 5C) Representative in vivo bioluminescent imaging signals and total flux (photons/s) (log₁₀ scale). (FIG. 5D) Mean total flux (photons/s)±SEM. (FIG. 5E-FIG. 5I) Skin tissue was harvested on day 3 post-infection from WT and PAD4^{-/-} mice for histology (n≥4 per group) and for flow cytometry analysis (n≥6 per group). (FIG. 5E) Representative H&E skin sections (Scale bars, 500 μm). Black arrows indicate the region of the horizontal area of neutrophilic abscess. (FIG. 5F) Mean neutrophilic abscess area (cm²)±SEM. (FIG. 5G) Total number of neutrophils, monocytes, and macrophages±SEM. (FIG. 5H) Representative Gram stain skin sections (Scale bars, 500 μm). (FIG. 5I) Mean bacterial band width (mm)±SEM. Results are representative of at least two independent experiments. †P<0.01, ‡P<0.001, as calculated by a two-way ANOVA test (FIG. 5B and FIG. 5D) or a Student's t test (2-tailed) (FIG. 5F, FIG. 5G, and FIG. 5I). ns, not significant;

FIG. 6A, FIG. 6B, FIG. 6C, FIG. 6D, FIG. 6E, FIG. 6F, and FIG. 6G demonstrate that TNFR2 is crucial for NET formation, NOX2 activation, and ROS production during S. aureus skin infections. S. aureus (3×10⁷ CFU) skin infections were performed on WT, TNF^{-/-}, TNFR1^{-/-}, TNFR2^{-/-}, and PAD4^{-/-} mice and the infected skin and kidneys were harvested on day 3 post-infection for histology (n≥6 per group), flow cytometry analysis (n≥8 per group), and CFU counts (n≥8 per group). (FIG. 6A) Representative immunofluorescence microscopy sections imaged at 400× magnification labeled with anti-citrullinated histone 3 (H3-Cit) (green), anti-Ly6G (red), and DAPI (blue) (Scale bars, 100 m). (FIG. 6B) Integrated density of H3-Cit±SEM. (FIG. 6C) Mean colony forming units (CFU) per kidney pair (log scale)±SEM. (FIG. 6D) Total number of suicidal and vital neutrophil extracellular traps (NETs)±SEM. (FIG. 6E) Mean of ROS mean fluorescence intensity (MFI)±SEM in neutrophils. (FIG. 6F) Representative immunofluorescence microscopy sections labeled with anti-phosphorylated p47 (phos-p47) (green), anti-Ly6G (red), and DAPI (blue) (Scale bars, 100 μm). (FIG. 6G) Mean count of phos-p47 puncta (count/mm²)±SEM. Results are combined from at least two independent experiments. *P<0.05, †P<0.01, ‡P<0.001, as calculated by one-way ANOVA multiple comparisons test (FIG. 6B-FIG. 6E, and FIG. 6G). ns, not significant;

FIG. 7A, FIG. 7B, FIG. 7C, FIG. 7D, FIG. 7E, FIG. 7F, FIG. 7G, FIG. 7H, FIG. 7I, FIG. 7J, and FIG. 7K show that TNFR2 agonist has efficacy against S. aureus skin infections in mice. Mice were treated with a single dose of vehicle (sterile PBS) or TNFR2 agonist (8 mg/kg, i.p.) (n≥8 mice per group) at 4 hours after S. aureus (3×10⁷ CFU) intradermal skin inoculation. (FIG. 7A) Representative photographs of skin lesions. (FIG. 7B) Mean total lesion size (cm²)±SEM. (FIG. 7C) Representative in vivo bioluminescence imaging (BLI). (FIG. 7D) Mean total flux (photons/s)±SEM. (FIG. 7E-FIG. 7G) Infected skin was harvested on day 3 post-infection from vehicle-treated or TNFR2 agonist-treated mice for flow cytometry analysis (n≥9 per group). (FIG. 7E) Total number of neutrophils±SEM. (FIG. 7F) Total number of suicidal and vital NETs±SEM. (FIG. 7G) Mean ROS mean fluorescence intensity (MFI)±SEM in neutrophils. (FIG. 7H-FIG. 7K) Mice were treated with a single dose of vehicle (sterile PBS) or TNFR2 agonist (8 mg/kg, i.p.) at 4 hours after S. aureus intradermal skin inoculation. Infected skin and kidneys were harvested on day 3 post-infection from vehicle-treated or TNFR2 agonist-treated mice for histology (n≥5 mice per group), flow cytometry analysis (n≥9 per group), and CFU counts (n≥5 per group). (FIG. 7H) Representative H&E skin sections (Scale bars, 500 μm). Black arrows indicate the region of the horizontal area of neutrophilic abscess. (FIG. 7I) Mean neutrophilic abscess area (cm²) A SEM. (FIG. 7J) Mean % of CD63+ neutrophils±SEM. (FIG. 7K) Mean colony forming units (CFU) per kidney pair (log scale)±SEM. *P<0.05, †P<0.01 and ‡P<0.001, as calculated by a two-way ANOVA test (FIG. 7B and FIG. 7D) or a Student's t test (2-tailed) (FIG. 7E. FIG. 7F, FIG. 7G. FIG. 7I), or a Mann-Whitney test (FIG. 7J). Data are representative (FIG. 7H. FIG. 7I and FIG. 7J) or a compilation of at least two independent experiments. ns, not significant;

FIG. 8A, FIG. 8B, FIG. 8C, FIG. 8D, FIG. 8E, and FIG. 8F show that TNFR2 agonist has efficacy against P. aeruginosa skin infections in mice. Mice were treated with a single dose of vehicle (sterile PBS) or TNFR2 agonist (8 mg/kg, i.p.) (n≥10 mice per group) at 4 hours after P. aeruginosa (1×10⁶ CFU) intradermal skin inoculation. (FIG. 8A) Representative photographs of skin lesions of P. aeruginosa infection. (FIG. 8B) Mean total lesion size (cm²) SEM of P. aeruginosa infection. (FIG. 8C) Representative in vivo bioluminescence imaging (BLI) of P. aeruginosa infection. (FIG. 8D) Mean total flux (photons/s)±SEM of P. aeruginosa infection. (FIG. 8E) Mean weight change (% of initial)±SEM. (FIG. 8F) Kaplan-Meier survival curves of vehicle- and TNFR2 agonist-treated mice after P. aeruginosa intradermal infection. *P<0.05, †P<0.01 and ‡P<0.001, as calculated by a two-way ANOVA test (FIG. 8B, FIG. 8D, FIG. 8F, and FIG. 8H). Data are a compilation of at least two independent experiments. ns, not significant; and

FIG. 9 is a model of TNFR-mediated neutrophil responses against S. aureus skin infection S. aureus intradermal infections result in TNF expression by recruited neutrophils in the skin. TNF acts directly on neutrophils by TNFR1 to increase their recruitment to the skin, whereas TNFR2 promoted NET formation, ROS production, NOX2 activation, and azurophilic degranulation. TNFR2 agonist treatment improved the immune response against bacterial skin infections by enhancing NET formation in the infected skin.

FIG. 10A, FIG. 10B, and FIG. 10C show bone marrow reconstitution of WT and TNF^{-/-} mice. (FIG. 10A) Survival rate of mice after irradiation of mice with 8 gy. (FIG. 10B) Representative flow plots for circulating CD45+ TNF+ cells (%) 8 weeks after bone marrow reconstitution. (FIG. 10C) Mean CD45+ TNF+ cells from live cells (%)±SEM.

FIG. 11 shows the gating strategy for TNF+, TNFR1+, and TNFR2+ cells in the skin. Single cells were isolated from naïve or infected skin at different time points of infection with S. aureus and stained with flow cytometric markers for analysis. Representative flow plots for TNF+, TNFR1+, and TNFR2+ monocytes, neutrophils, and macrophages day 3 infected WT skin.

FIG. 12A, FIG. 12B and FIG. 12C show neutrophil adoptive transfer into WT, TNFR1^{-/-}, and TNFR2^{-/-} mice. Neutrophils were isolated from 8-10 week old WT, TNFR1^{-/-}, and TNFR2^{-/-} mice. Representative flow plots of isolated neutrophils from WT (FIG. 12A), TNFR^{-/-} (FIG. 12B), and TNFR2^{-/-} (FIG. 12C) for viability and purity before transfer into recipient mice.

FIG. 13 shows the gating strategy for myeloid cell populations and ROS+ neutrophils in the skin. Single cells were isolated from naïve or infected skin at different time points of infection with S. aureus and stained with flow cytometric markers for analysis. Representative flow plots showing the gating strategy for monocytes, neutrophils, and macrophages and neutrophil-derived ROS from day 3 infected WT skin.

FIG. 14A and FIG. 14B show the gating strategy for suicidal and vital NETs. (FIG. 14A) Representative flow plots showing the gating strategy for suicidal and vital NETs. (FIG. 14B) Representative flow plots of suicidal and vital NETs from WT, TNF^{-/-}, TNFR1^{-/-}, TNFR2^{-/-}, and PAD4^{-/-} infected day 3 skin.

FIG. 15A, FIG. 15B and FIG. 15C show TNFR2 is critical for the induction of NETs upon in vitro exposure to S. aureus. After 2 hours of incubation with R1 medium (−). PMA, live S. aureus at MOI 100, neutrophils were stained with Sytox Green and analyzed by flow cytometry analysis. (FIG. 15A) Representative flow plots showing the gating strategy for suicidal and vital NETs. (FIG. 15B and FIG. 15C) Mean of suicidal NETs (FIG. 15B) and vital NETs (FIG. 15C) mean fluorescence intensity (MFI)±SEM. *P<0.05, as calculated by Dunnett's one-way ANOVA multiple comparisons test (FIG. 15B and FIG. 15C). ns, not significant. Results are representative of two independent experiments.

FIG. 16A and FIG. 16B show TNFR1 and TNFR2 are critical for neutrophil production of ROS upon in vitro stimulation with S. aureus. After 2 hours of incubation with RPMI supplemented with 10% FBS (−), PMA, live S. aureus at MOI 100, neutrophils were stained with DCFDA dye and analyzed by flow cytometry analysis. (FIG. 16A) Representative flow plots showing the gating strategy for ROS production. (FIG. 16B) Mean of ROS mean fluorescence intensity (MFI)±SEM. *P<0.05, †P<0.01, ‡P<0.001, as calculated by Dunnett's one-way ANOVA multiple comparisons test. ns, not significant. Results are representative of two independent experiments.

FIG. 17A and FIG. 17B show the TNFR2 agonist does not have direct antibacterial activity against S. aureus and P. aeruginosa. Bacterial broth cultures were incubated with vehicle (0 μg/mL) or various concentrations of TNFR2 agonist (80 μg/mL, 160 μg/mL, and 320 μg/mL. The bacterial growth (OD600) was measured in triplicate (n=3) for 10 hours with measurements recorded at 20-minute intervals. Bacterial growth (OD600) in S. aureus (FIG. 17A) and P. aeruginosa (FIG. 17B).

FIG. 18A, FIG. 18B, FIG. 18C, FIG. 18D. FIG. 18E, FIG. 18F. FIG. 18G, FIG. 18H. FIG. 18I, and FIG. 18J show that TNF does not regulate chemokine, cytokine, and antimicrobial peptide expression in S. aureus-infected skin. S. aureus skin infections were performed on WT and TNF^{-/-} mice and the infected skin tissue was harvested on day 3 post-infection for qRT-PCR analysis (n≥5 per group). Mean mRNA levels (Arbitrary Units [A.U.])±s.e.m. of transcripts for (FIG. 18A-FIG. 18D) chemokines (FIG. 18A) Cxcl1, (FIG. 18B) Cxcl2, (FIG. 18C) Cxcl3, and (FIG. 18D) Cxcl5; (FIG. 18E-FIG. 18G) antimicrobial peptides (FIG. 18E) Mpo, (FIG. 18F) S100a8, and (FIG. 18G) S100a9; (FIG. 18H-FIG. 18J) proinflammatory cytokines (FIG. 18H) I/1b, (FIG. 18I) 116, and (FIG. 18J) Tnf. Results are representative of at least two independent experiments.*P<0.05, as calculated by a Student's t-test (2-tailed). ns, not significant.

FIG. 19A and FIG. 19B show that TNF does not regulate IL-1β expression in S. aureus-infected skin. S. aureus skin infections were performed on WT and TNF^{-/-} mice and the infected skin tissue was harvested on day 3 post-infection for flow cytometry analysis (n≥4 per group). (FIG. 19A) Total number of pro-IL-1β+ neutrophils, monocytes, and macrophages±SEM. (FIG. 19B) Mean fluorescence intensity of pro-IL-1β in neutrophils±SEM. Results are representative of at least two independent experiments. Statistics calculated by a Student's t-test (2-tailed). ns, not significant.

FIG. 20A, FIG. 20B, FIG. 20C, FIG. 20D. FIG. 20E, and FIG. 20F show that TNF promotes IL-17A expression by γδ+ and CD4+ T cells. S. aureus skin infections were performed on WT and TNF^{-/-} mice and the infected skin tissue and in the draining lymph nodes (dLNs) were harvested on day 7 post-infection for flow cytometry analysis (n≥7 per group). (FIG. 20A and FIG. 20D) Total number of γδ+ and CD+ T cells±SEM in the skin (FIG. 20A) and in the dLNs (FIG. 20D). (FIG. 20B and FIG. 20E) Representative flow plots for IL-17A+γδ+ and CD+ T cells in the skin (FIG. 20B) and in the dLNs (FIG. 20E). (FIG. 20C and FIG. 20F) Total number of IL-17A+γδ+ and CD+ T cells A SEM in the skin (FIG. 20C) and in the dLNs (FIG. 20F). Results are combined from at least two independent experiments. *P<0.05, as calculated by a Student's r test (2-tailed) (FIG. 20B and FIG. 20D). ns, not significant.

FIG. 21A, FIG. 21B. FIG. 21C, and FIG. 21D show that adoptive transfer of WT neutrophils does not rescue the immune defect in TNF^{-/-} mice. Neutrophils from TNF^{-/-} or WT donor mice were adoptively transferred into TNF^{-/-} mice (n≥6 per group). After 2 hours, the recipient mice were infected intradermally with S. aureus. (FIG. 21A) Representative photographs of skin lesions. (FIG. 21B) Mean total lesion size (cm²) SEM. (FIG. 21C) Representative in vivo bioluminescent imaging signals and total flux (photons/s) (log₁₀ scale). (FIG. 21D) Mean total flux (photons/s)±SEM. Results are combined from two independent experiments. Statistics calculated by a two-way ANOVA test (FIG. 21B and FIG. 21D). ns, not significant.

FIG. 22A and FIG. 22B show that TNFR2 is crucial for azurophilic degranulation of neutrophils in the skin. S. aureus skin infections were performed on WT, TNFR1^{-/-}, TNFR2^{-/-}, and PAD4^{-/-} mice and the blood and infected skin biopsies were harvested on day 3 post-infection for flow cytometry analysis of azurophilic (CD63+) and secretory (CD35+) degranulation (n≥5 per group). (FIG. 22A) Mean % of CD63+ neutrophils±SEM. (FIG. 22B) Mean % of CD35+ neutrophils±SEM. *P<0.05, †P<0.01, as calculated by one-way ANOVA multiple comparisons test. ns, not significant.

FIG. 23A, FIG. 23B. FIG. 23C, FIG. 23D, FIG. 23E, and FIG. 23F show that TNFR2 regulates gene and protein expression during S. aureus skin infections. S. aureus skin infections were performed on WT and TNFR2^{-/-} mice and the infected skin biopsies were harvested on day 3 post-infection for RNA sequencing (n≥5 per group). (FIG. 23A) Volcano plot with the log 2 fold changes in gene expression between WT and TNFR^{-/-} mice after infection on the x-axis and the statistical significance (−log 10 p value) on the y-axis. The gene symbols of 3 annotated genes that are most significantly different in gene expression are displayed. (FIG. 23B) FPKMs of top 3 downregulated genes in TNFR2^{-/-} mice in comparison to WT mice S.-aureus-infected skin. (FIG. 23C) FPKMs of top 3 upregulated genes in TNFR2^{-/-} mice in comparison to WT mice S.-aureus-infected skin. (FIG. 23D and FIG. 23E) Infected skin tissue and serum were harvested from WT mice on days 0 through 7 for TNF protein quantification (n≥4). (FIG. 23D) Mean TNF protein levels (picograms/milligram of tissue)±SEM from S. aureus infected skin. (FIG. 23E) Mean TNF protein levels in serum (picograms/milliliter of serum)±SEM from S. aureus infected mice. (FIG. 23F) SDS-PAGE gel of protein isolated from 1.0×10⁴ neutrophils from WT and TNFR2^{-/-} mice. *P<0.05, †P<0.01, ‡P<0.001, as calculated by Student's t test (2-tailed test) (FIG. 23B and FIG. 23C) and a one-way ANOVA multiple comparisons test (FIG. 23D and FIG. 23E). ns, not significant.

FIG. 24A and FIG. 24B show that blocking TNFR2 impairs host protection against S. aureus skin infections. S. aureus skin infections were performed on WT mice treated with an α-TNFR2 antibody or isotype control every other day beginning at d−1 and throughout the infection. (FIG. 24A) Mean total lesion size (cm²)±SEM. (FIG. 24B) Representative in vivo bioluminescent imaging signals and total flux (photons/s) (log₁₀ scale). ‡P<0.001, as calculated by a two-way ANOVA test. ns, not significant.

FIG. 25A and FIG. 25B show that TNFR2 signaling promotes granularity in neutrophils. S. aureus (3×10⁷ CFU) skin infections were performed on WT (FIG. 25A), vehicle and TNFR2 agonist treated (FIG. 25B) mice and the infected skin harvested on day 3 after infection for flow cytometry. (FIG. 25A) Neutrophils from WT infected skin were gated on TNFR2− and TNFR2+ expression by flow cytometry and side scatter (SSC) mean fluorescence intensity (MFI) measured. (FIG. 25B) Neutrophils from day 3 infected vehicle and TNFR2 agonist-treated mice blood and skin were isolated and SSC MFI±SEM measured by flow cytometry. **P<0.01, ****P<0.0001, as calculated by a Student's t test (two-tailed).

### DETAILED DESCRIPTION

The presently disclosed subject matter now will be described more fully hereinafter with reference to the accompanying Figures, in which some, but not all embodiments of the inventions are shown. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### A. Bacterial Skin Infections

Bacterial skin infections can develop when bacteria enter the body through cuts, open wounds, or other breaks in the skin, for example, breaks in the skin that result from scrapes, punctures, surgery, burns, sunburn, animal or insect bites, wounds, and preexisting skin disorders. Symptoms may include redness, swelling, pain, or pus. Bacterial skin infections can be mild and easily treatable; however, some can become very serious and even life threatening. Bacterial skin infections can range in size from a tiny spot to the entire body surface. Additionally, some bacterial infections can be spread to others. The most common bacteria that can infect the skin are Staphylococcus and Streptococcus.

Staphylococcus aureus (S. aureus) is a gram-positive bacterium and is the leading cause of skin and soft tissue infections (SSTI) as well as invasive infections causing endocarditis, osteomyelitis, and sepsis (Reference 1). The emergence of antibiotic-resistant S. aureus is a serious health burden, as methicillin-resistant S. aureus (MRSA) constitutes the majority of clinical isolates in the U.S. and is a predominant cause of death due to antibiotic-resistant bacteria worldwide (Reference 2). Furthermore, SSTIs caused by MRSA account for more than 14 million outpatient and emergency room visits and greater than 750,000 hospital admissions in the U.S. annually (References 3, 4). With the emergence of antibiotic-resistant S. aureus outpacing the development of novel antibiotics, there is an urgent need to better understand the protective immune mechanisms against S. aureus infections for the development of host-directed therapeutics as alternatives to antibiotics.

### B. Tumor Necrosis Factor (TNF) Signaling and Treatment of Infections and Inflammatory Diseases

Tumor necrosis factor (TNF) is a pleiotropic cytokine that signals through its two cognate receptors, TNF receptor 1 (TNFR1) and TNF receptor 2 (TNFR2) (References 5, 6). TNFR1 is constitutively expressed by the majority of nucleated cells, whereas TNFR2 expression is induced upon activation and restricted to specific immune cells (e.g., regulatory T cells (Tregs), B cells, and macrophages) and non-immune cells (e.g., endothelial cells, epithelial cells, and fibroblasts) (References 7-12). TNF is involved in the pathogenesis of autoimmune and inflammatory diseases, such as rheumatoid arthritis, irritable bowel syndrome, psoriasis, psoriatic arthritis, and ankylosing spondylitis (Reference 7), which has led to the development of Food and Drug Administration (FDA)-approved TNF inhibitors to treat these disorders (Reference 13). Notably, TNF inhibitor-treated patients have an increased risk of S. aureus skin colonization and infection (References 14, 15), suggesting the involvement of TNF in protection against S. aureus in the skin. The TNF/NFR-mediated mechanisms of host defense against S. aureus skin infections, however, are not entirely understood.

The presently disclosed subject matter provides insight into the role of TNF and the differential contributions of TNFR1 and TNFR2 in protection against skin infections, such as S. aureus skin infections. As shown herein, in an in vivo mouse model of S. aureus intradermal infection, TNF promoted protection against S. aureus in the skin, which was mediated by bone marrow-derived immune cells. Furthermore, neutrophil-intrinsic TNFR1 and TNFR2 signaling directed immunity against S. aureus skin infections. Mechanistically, TNFR1 promoted neutrophil recruitment to the skin, whereas TNFR2 prevented systemic bacterial dissemination and directed neutrophil function, including NOX2 activation, ROS production, and PAD4-dependent neutrophil extracellular trap (NET) formation. Also as shown herein, treatment with a TNFR2 agonist showed therapeutic efficacy against skin infections.

Provided herein are treatment methods that use TNFR2 modulating agents. As used herein, “TNFR2 modulating agent” refers to a composition that affects TNFR2 function and/or signaling. In some embodiments, a TNFR2 modulating agent inhibits TNFR2 function and/or signaling. In some embodiments, a TNFR2 modulating agent promotes (e.g., enhances, increases, prolongs, and the like) TNFR2 function and/or signaling. In some embodiments, a TNFR2 modulating agent is a TNFR2 agonist. In some embodiments, a TNFR2 modulating agent is a TNFR2 antagonist.

In some embodiments, a TNFR2 modulating agent is an antibody or antibody fragment that binds to human TNFR2. In some embodiments, a TNFR2 modulating agent is an agonistic or antagonistic anti-TNFR2 antibody, such as an agonistic or antagonistic anti-TNFR2 antibody described in WO 2022/147222 A1.

In some embodiments, the presently disclosed subject matter provides a method for treating a bacterial skin infection in a subject, the method comprising administering to a subject in need of treatment thereof a therapeutically effective amount of a TNFR2 agonist.

In certain embodiments, the TNFR2 agonist is an antibody, for example a monoclonal antibody (mAb). In some embodiments, the TNFR2 agonist is a humanized mAb. In some embodiments, the TNFR2 agonist is a fully human mAb. In some embodiments, the TNFR2 agonist is a human IgG1 mAb. In some embodiments, the TNFR2 agonist is in preclinical development or under clinical trial, such as TNFR2 antibodies described in Bai J, Ding B and Li H (2022) Targeting TNFR2 in Cancer, All Roads Lead to Rome. Front. Immunol. 13:844931. In some embodiments, the TNFR2 agonist is selected from an antibody in Table 1. In some embodiments, the TNFR2 agonist is selected from the group consisting of BI-1910, HFB3200301 and MM-401, which are listed in Table 1.

TABLE 1
TNFR2 antibodies

| Agonist/Antagonist | Name | Reference |
|---|---|---|
| Antagonist | BI-1808 | BI-1808 as a Single Agent and With Pembrolizumab in |
| | | Treatment of Advanced Malignancies. Available at: |
| | | ClinicalTrials.gov/show/NCT04752826. |
| | | Mårtensson L, Kovacek M, Holmkvist P, Semmrich M, |
| | | Svensson C, Blidberg T, et al. 725 Pre-Clinical Development of |
| | | TNFR2 Ligand-Blocking BI-1808 for Cancer Immunotherapy. |
| | | J ImmunoTherapy Cancer (2020) 8: A768-8. doi: 10.1136/jitc- |
| | | 2020-SITC2020.0725 |
| Antagonist | BITR2101 | Boston Immune Technologies and Therapeutics and Beigene |
| | (also | Enter Into an Exclusive Option and License Agreement to |
| | referred | Develop Novel TNFR2 Antagonists. |
| | to as | Available at: www.bostonimmunetech.com/media/#tnf. |
| | BITT-2101) | www.businesswire.com/news/home/20230818088156/en/BITT- |
| | | Announces-FDA-Acceptance-of-IND-for-TNFR2-Antibody |
| Antagonist | APX601 | Krishnan S, Alvarado R, Huang G, Yang X, Filbert E L. |
| | | Abstract LB175: APX601, a Potent TNFR2 Antagonist as a |
| | | Novel and Promising Approach to Reverse Tumor Immune |
| | | Suppression. Cancer Res (2021) 81(13 Supplement): |
| | | LB175. doi: 10.1158/1538-7445.AM2021-LB175 |
| | | Filbert E, Krishnan S, Alvarado R, Huang G, Bahjat F, Yang X. |
| | | 693 APX601, a Novel TNFR2 Antagonist Antibody for Cancer |
| | | Immunotherapy. J ImmunoTher of Cancer (2020) 8(Suppl 3): |
| | | A417. doi: 10.1136/jitc-2020- SITC2020.0693 |
| Antagonist | AN3025 | Chen Y, Jia M, Xu S, Zhao Y, Chan E, Zhang M, et al. Abstract |
| | | 1451: AN3025: A Novel Anti-Human TNFR2 Antibody That |
| | | Exhibits Immune Activation |
| | | and Strong Anti-Tumor Activity In Vivo. Cancer Res (2021) |
| | | 81(13 Supplement): 1451. doi: 10.1158/1538-7445.AM2021-1451 |
| Antagonist | SIM0235 | IND Application for Simcere's Independently Developed Anti- |
| | (also | TNFR2 Antibody Accepted by CDE. Available at: |
| | referred | en.simcere.com/news/detail.aspx?mtt=170 |
| | to as | |
| | SIM1811-03) | |
| Antagonist | LBL-019 | Available at: en.leadsbiolabs.com/R-D-pipeline.html |
| | (also | |
| | referred to | |
| | as BLB-019) | |
| Antagonist | NBL-020 | Sum C S, Danton M, Pritsker A, Lin R, Yu R, et al. Abstract |
| | | 1869: Novel TNFR2 Antibodies to Overcome T Cell |
| | | Exhaustion and Suppressive Tumor Microenvironment. Cancer |
| | | Res (2021) 81(13 Supplement): 1869. doi: 10.1158/1538- |
| | | 7445.AM2021-1869 |
| Agonist | BI-1910 | Mårtensson L, Cleary K, Semmrich M, Kovacek M, Holmkvist |
| | | P, Svensson C, et al. Abstract 936: Targeting TNFR2 for |
| | | Cancer Immunotherapy: Ligand Blocking Depletors Versus |
| | | Receptor Agonists. Cancer Res (2020) 80(16 Supplement): 936. |
| | | doi: 10.1158/1538-7445.AM2020-936 |
| Agonist | HFB200301 | Wei S, Fulton R, Lu Y-Y, Zhang Q, Zhou H, Raue A, et al. |
| | | Abstract 1883: Mechanism of Action and Biomarker Strategy |
| | | for HFB200301, an Anti- TNFR2 Agonist Antibody for the |
| | | Treatment of Cancer. Cancer Res (2021) 81 (13 Supplement): |
| | | 1883-3. doi: 10.1158/1538-7445.AM2021-1883 |
| Agonist | MM-401 | Sampson J F, Kurella V B, Paragas V, Kumar S, Lulo J E, Qiu |
| | | J A, et al. Abstract 555: A Novel Human TNFR2 Antibody |
| | | (MM-401) Modulates T Cell Responses in Anti-Cancer |
| | | Immunity. Cancer Res (2019) 79(13 Supplement): 555. doi: |
| | | 10.1158/1538-7445.AM2019-555 |
| | | Richards J, Wong C, Koshkaryev A, Fulton R, Camblin A, |
| | | Sampson J, et al. Abstract 4846: MM-401, a Novel Anti- |
| | | TNFR2 Antibody That Induces T Cell Co-Stimulation, Robust |
| | | Anti-Tumor Activity and Immune Memory. Cancer Res (2019) |
| | | 79(13 Supplement): 4846. doi: 10.1158/1538-7445.AM2019-4846 |

The HiFiBio candidate, HFB200301, is a humanized anti-TNFR2 antibody, does not compete with TNF for TNFR2 binding, stimulates activated CD4 and CD8 T cells and enhances their proliferation in vitro, and displays Fc receptor-independent anti-tumor activity in a syngeneic MC38 tumor model in human TNFR2 knock-in mice (Wei et al., AACR 2020, Poster #2282).

The Bioinvent candidate B1-1910 is characterized by strong activation of TNFR2 signaling, does not require Fc engagement for biological activity, but shows enhanced activity as an IgG isotype or variant Fc regions designed to improve binding to inhibitory as opposed to activating FcyR. WO 2020/089473, filed by Bioinvent, describes agonistic anti-TNFR2 antibodies and indicates that the agonist antibodies seem to bind to the distal C-terminal part of CRD3 and that binding likely depends on to a greater extent on CRD4 than antagonistic anti-TNFR2 antibodies evaluated in the same epitope mapping experiments. Treatment with a murine surrogate of BI-1910 (i.e., antibody 5A05 in a murine IgG1 format) antibody resulted in an early increase in intratumoral CD8 T cells in a CT26 syngeneic model resulting in an enhanced CD8/Treg ratio (Martensson, et al AACR 2020, Abstract #936).

Merrimack's anti-TNFR2 antibody candidate, MM-401, binds to the same epitope as murine antibody Y9 (described in Tam et al., Sci. Transl. Med., 11(512), 2019 as binding to an epitope in CRD1). Antitumor efficacy in mouse syngeneic tumor models is FcyR dependent and enhanced by engagement of inhibitory FcyRs (Richards et al, MM-401, a novel anti-TNFR2 antibody that induces T cell co-stimulation. AACR 2019, Abstract #4848).

In certain embodiments, the TNFR2 agonist is a ligand-based TNFR2 agonist. In some embodiments, the TNFR2 agonist is a single-chain ligand based TNFR2-specific agonist. In some embodiments, the TNFR2 agonist is a multimeric ligand-based agonist of TNFR2. In some embodiments, the agonist is any one of the TNFR2 agonists described or referenced in Vargas J G et al. (Front. Immunol., 2022). In some embodiments, the agonist is (irr(mu)IgG1(D265A)-HC:sc(mu)TNF80), as described in Vargas J G et al. (Front. Immunol., 2022). In some embodiments, the agonist is STAR2 or NewSTAR2, as described in Vargas J G et al. (Front. Immunol., 2022).

In certain embodiments, the TNFR2 agonist is a small molecule.

In some embodiments, the presently disclosed subject matter provides a composition comprising a TNFR2 agonist and a pharmaceutically acceptable carrier.

In certain embodiments, the TNFR2 agonist inhibits (e.g., blocks, partially blocks, interferes, decreases, or reduces) the growth of bacteria or a bacterial infection. Thus, one of ordinary skill in the art would appreciate that the term “Inhibit” encompasses a complete and/or partial decrease in the growth of bacteria or a bacterial infection, e.g., a decrease by at least 10%, in some embodiments, a decrease by at least 20%, 30%, 50%, 75%, 95%, 98%, and up to and including 100%.

In some embodiments, the presently disclosed subject matter provides a method for inhibiting the growth of bacteria or a bacterial infection, comprising administering a therapeutically effective amount of a TNFR2 agonist to a subject in need thereof. In some embodiments, the subject has a bacterial skin infection or is at risk of developing a bacterial skin infection.

In some embodiments, the presently disclosed subject matter provides a TNFR2 agonist for use in the treatment of a bacterial skin infection in a subject in need of treatment thereof.

In some embodiments, the presently disclosed subject matter provides a composition comprising a TNFR2 agonist for use in a method for the treatment of a bacterial skin infection.

In some embodiments, the presently disclosed subject matter provides the use of a TNFR2 agonist for the manufacture of a medicament for the treatment of a bacterial skin infection.

In some embodiments, administering of a therapeutically effective amount of a TNFR2 agonist promotes wound healing, reduces the size of one or more skin lesions formed after a bacterial infection, accelerates the rate of reduction in size of one or more skin lesions after a bacterial infection, reduces bacterial burden in a subject and/or at one or more sites of bacterial infection, and/or promotes bacterial clearance after infection. Methods of determining bacterial burden and clearance are well known in the art. For example, guidance for physicians for testing and diagnosing infectious diseases is provided in Miller, J. M. et al. A Guide to Utilization of the Microbiology Laboratory for Diagnosis of Infectious Diseases: 2018 Update by the Infectious Diseases Society of America and the American Society for Microbiology. Clinical Infectious Diseases, Volume 67, Issue 6, 31 Aug. 2018, Pages e1-e94. In some embodiments, administering of a therapeutically effective amount of a TNFR2 agonist promotes wound and/or skin lesion healing, which includes reducing the size of one or more skin lesions, accelerating the rate of reduction in size of one or more skin lesions, reducing pain, reducing firmness around the infection, decreasing swelling, diminished redness, fading of any discoloration, and combinations thereof.

In certain embodiments, the administering of a therapeutically effective amount of a TNFR2 agonist controls bacterial spread from the skin.

In some embodiments, the presently disclosed subject matter provides a method for controlling bacterial spread from the skin, comprising administering a therapeutically effective amount of a TNFR2 agonist to a subject in need thereof.

In certain embodiments, the administering of a therapeutically effective amount of a TNFR2 agonist inhibits (e.g., reduces or prevents) systemic dissemination of a bacterial skin infection.

In some embodiments, the presently disclosed subject matter provides a method for inhibiting systemic dissemination of a bacterial skin infection, comprising administering a therapeutically effective amount of a TNFR2 agonist to a subject in need thereof. In some embodiments the method further comprises administering one or more additional agents for inhibiting systemic dissemination of a bacterial skin infection. For example, the one or more additional agents is an antibiotic.

In certain embodiments, the administering of a therapeutically effective amount of a TNFR2 agonist improves pathogen clearance. In certain embodiments, the administering of a therapeutically effective amount of a TNFR2 agonist improves clearance of S. aureus and P. aeruginosa skin infections. In certain embodiments, improved clearance of pathogenic skin infections that is mediated by a TNFR2 agonist involves increased NET formation at the infection site.

In certain embodiments, the administering of a therapeutically effective amount of a TNFR2 agonist promotes neutrophil extracellular trap (NET) formation. In particular embodiments, the TNFR2 agonist promotes increased NET formation at the infection site. In particular embodiments, the TNFR2 agonist enhances vital NETosis. In particular embodiments, the TNFR2 agonist enhances suicidal NETosis.

In some embodiments, the presently disclosed subject matter provides a method for promoting increased NET formation at an infection site, comprising administering to a subject in need thereof a therapeutically effective amount of a TNFR2 agonist. In some embodiments, the infection site is a bacterial infection site. For example, the infection site is a skin infection site. In some embodiments, the bacteria is S. aureus. In some embodiments, the bacteria is P. aeruginosa. In some embodiments, the TNFR2 agonist enhances vital NETosis. In some embodiments, the TNFR2 agonist enhances suicidal NETosis.

In some embodiments, the TNFR2 agonist is administered after a bacterial skin infection occurs. In some embodiments, the TNFR2 agonist is administered after diagnosis of the bacterial skin infection. In some embodiments, the TNFR2 agonist is administered after treatment with an antibiotic. In some embodiments, the TNFR2 agonist is administered if treatment with an antibiotic is not possible, for example if the bacterial infection is resistant to antibiotic treatment. In some embodiments, the TNFR2 agonist is administered to a subject infected with an antibiotic resistant pathogen. In some embodiments, the antibiotic resistant pathogen is methicillin-resistant S. aureus.

In some embodiments, the TNFR2 agonist is administered at one or more site(s) of bacterial skin infection.

In some embodiments, the TNFR2 agonist is administered within hours of a bacterial infection. In some embodiments, the TNFR2 agonist is administered within 1, 2, 3, 4, 5, 6, 7, 8, 9, 10, 11, 12, 13, 14, 15, 16, 17, 18, 19, 20, 21, 22, 23, or 24 hour(s) after a bacterial infection. For example, the TNFR2 agonists is administered within 24 hours after a bacterial infection. In some embodiments, the TNFR2 agonist is administered within days of a bacterial infection. In some embodiments, the TNFR2 agonist is administered within 1, 2, 3, 4, 5, 6, 7, 8, 9, 10, 11, 12, 13, or 14 day(s) after a bacterial infection. For example, the TNFR2 agonists is administered within 3 days of bacterial infection.

In some embodiments, the TNFR2 agonist is administered in multiple doses.

In some embodiments, the presently disclosed subject matter provides a method for treating an inflammatory disease associated with neutrophil extracellular trap (NET) formation, the method comprising administering to a subject in need of treatment thereof a therapeutically effective amount of a TNFR2 antagonist. In certain embodiments, the inflammatory disease is exacerbated by NET formation. NETs have been implicated in the development of autoimmune inflammatory diseases such as psoriasis (References 109, 110), atherosclerosis (Reference 111), and systemic lupus (References 112, 113). In certain embodiments, the inflammatory disease is an autoimmune inflammatory disease such as psoriasis, atherosclerosis, or systemic lupus. In some embodiments, the TNFR2 antagonist is an antibody, for example a monoclonal antibody (mAb). In some embodiments, the TNFR2 antagonist is a humanized mAb. In some embodiments, the TNFR2 antagonist is a fully human mAb. In some embodiments, the TNFR2 antagonist is a human IgG1 mAb. In some embodiments, the TNFR2 antagonist is in preclinical development or under clinical trial, such as TNFR2 antibodies described in Bai J, Ding B and Li H (2022) Targeting TNFR2 in Cancer: All Roads Lead to Rome. Front. Immunol. 13:844931. In some embodiments, the TNFR2 antagonist is selected from an antibody in Table 1.

In some embodiments, the presently disclosed subject matter provides a TNFR2 antagonist for use in the treatment of an inflammatory disease associated with neutrophil extracellular trap (NET) formation.

In some embodiments, the presently disclosed subject matter provides a composition comprising a TNFR2 antagonist for use in a method for the treatment of an inflammatory disease associated with NET formation.

In some embodiments, the presently disclosed subject matter provides the use of a TNFR2 antagonist for the manufacture of a medicament for the treatment of an inflammatory disease associated with NET formation.

In certain embodiments, the TNFR2 antagonist is a ligand-based TNFR2 antagonist. In some embodiments, the TNFR2 antagonist is a single-chain ligand based TNFR2-specific antagonist. In some embodiments, the TNFR2 antagonist is a multimeric ligand-based antagonist of TNFR2.

In certain embodiments, the TNFR2 antagonist is a small molecule.

In some embodiments, the presently disclosed subject matter provides a composition comprising a TNFR2 antagonist and a pharmaceutically acceptable carrier.

In certain embodiments, the administering of a therapeutically effective amount of a TNFR2 antagonist reduces NET formation. In certain embodiments, the administering of a therapeutically effective amount of a TNFR2 antagonist reduces inflammation.

In some embodiments, the presently disclosed subject matter provides a method for reducing NET formation, comprising administering to a subject in need thereof a therapeutically effective amount of a TNFR2 antagonist. In some embodiments, the subject suffers from or is diagnosed with an inflammatory disease that is exacerbated by NET formation. In some embodiments, the subject suffers from or is diagnosed with psoriasis, rheumatoid arthritis, and neutrophilic asthma. In some embodiments, the subject suffers from or is diagnosed with an autoimmune inflammatory disease. NETs have been implicated in the development of autoimmune inflammatory diseases such as psoriasis (References 109, 110), atherosclerosis (Reference 111), and systemic lupus (References 112, 113).

In some embodiments, the presently disclosed subject matter provides a TNFR2 antagonist for use in reducing NET formation in a subject in need thereof.

In some embodiments, the TNFR2 agonist is administered in combination with one or more other therapeutic agent(s). In some embodiments, the one or more other therapeutic agent(s) may be an antibiotic. In some embodiments, the one or more other therapeutic agent(s) is a TNFR1 agonist.

The term “combination” is used in its broadest sense and means that a subject is administered at least two agents, more particularly an agent described herein and at least one other therapeutic agent. More particularly, the term “in combination” refers to the concomitant administration of two (or more) active agents for the treatment of a, e.g., single disease state. As used herein, the active agents may be combined and administered in a single dosage form, may be administered as separate dosage forms at the same time, or may be administered as separate dosage forms that are administered alternately or sequentially on the same or separate days. In one embodiment of the presently disclosed subject matter, the active agents are combined and administered in a single dosage form. In another embodiment, the active agents are administered in separate dosage forms (e.g., wherein it is desirable to vary the amount of one but not the other). The single dosage form may include additional active agents for the treatment of the disease state.

Further, the agents described herein can be administered alone or in combination with adjuvants that enhance stability of the compounds, alone or in combination with one or more therapeutic agents, facilitate administration of pharmaceutical compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies.

The timing of administration of an agent described herein and at least one additional therapeutic agent can be varied so long as the beneficial effects of the combination of these agents are achieved. Accordingly, the phrase “in combination with” refers to the administration of an agent described herein and at least one additional therapeutic agent either simultaneously, sequentially, or a combination thereof. Therefore, a subject administered a combination of an agent described herein and at least one additional therapeutic agent can receive one agent and at least one additional therapeutic agent at the same time (i.e., simultaneously) or at different times (i.e., sequentially, in either order, on the same day or on different days), so long as the effect of the combination of both agents is achieved in the subject.

When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the agent described herein and at least one additional therapeutic agent are administered simultaneously, they can be administered to the subject as separate pharmaceutical compositions, each comprising either one agent or at least one additional therapeutic agent, or they can be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

In some embodiments, when administered in combination, the two or more agents can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of an agent described herein and at least one additional therapeutic agent is greater than the sum of the biological activities of the respective agents when administered individually.

Synergy can be expressed in terms of a “Synergy Index (SI),” which generally can be determined by the method described by F. C. Kull et al., Applied Microbiology 9, 538 (1961), from the ratio determined by:

$${Q}_{a} / {Q}_{A} + {Q}_{b} / {Q}_{B} = Synergy Index ( SI )$$

wherein:
Q_{A} is the concentration of a component A, acting alone, which produced an end point in relation to component A;
Qₐ is the concentration of component A, in a mixture, which produced an end point;
Q_{B} is the concentration of a component B, acting alone, which produced an end point in relation to component B; and
Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the S1, the greater the synergy shown by that particular mixture. Thus, a “synergistic combination” has an activity higher than what can be expected based on the observed activities of the individual components when used alone. Further, a “synergistically effective amount” of a component refers to the amount of the component necessary to elicit a synergistic effect in, for example, another therapeutic agent present in the composition.

### C. Pharmaceutical Compositions and Administration

Depending on the specific condition(s) being treated, TNFR2 modulating agents may be formulated into liquid or solid dosage forms and administered systemically or locally. The agents may be delivered, for example, in a timed- or sustained-slow release form as is known to those skilled in the art. Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articullar, intra-sternal, intra-synovial, intra-hepatic, intralesional, intracranial, intraperitoneal, intranasal, or intraocular injections or other modes of delivery.

For injection, the agents of the disclosure may be formulated and diluted in aqueous solutions, such as in physiologically compatible buffers such as Hank's solution, Ringer's solution, or physiological saline buffer. For such transmucosal administration, penetrants appropriate to the barrier to be permeated are used in the formulation. Such penetrants are generally known in the art.

Use of pharmaceutically acceptable inert carriers to formulate the TNFR2 modulating agents for the practice of the disclosure into dosages suitable for systemic administration is within the scope of the disclosure. With proper choice of carrier and suitable manufacturing practice, the compositions of the present disclosure, in particular, those formulated as solutions, may be administered parenterally, such as by intravenous injection. The agents can be formulated readily using pharmaceutically acceptable carriers well known in the art into dosages suitable for oral administration. Such carriers enable the agents of the disclosure to be formulated as tablets, pills, capsules, liquids, gels, syrups, slurries, suspensions and the like, for oral ingestion by a subject (e.g., patient) to be treated.

Pharmaceutical compositions suitable for use in the present disclosure include compositions wherein the active ingredients are contained in an effective amount to achieve its intended purpose. Determination of the effective amounts is well within the capability of those skilled in the art, especially in light of the detailed disclosure provided herein. The exact dosage will depend upon the route of administration, the form in which the agent is administered, the subject to be treated, the body weight of the subject to be treated, the bioavailability of the agent(s), the adsorption, distribution, metabolism, and excretion (ADME) toxicity of the agent(s), and the preference and experience of the attending physician.

In addition to the active ingredients, these pharmaceutical compositions may contain suitable pharmaceutically acceptable carriers comprising excipients and auxiliaries which facilitate processing of the active agents into preparations which can be used pharmaceutically. The preparations formulated for oral administration may be in the form of tablets, dragees, capsules, or solutions.

As used herein, the active agents may be combined and administered in a single dosage form, may be administered as separate dosage forms at the same time, or may be administered as separate dosage forms that are administered alternately or sequentially on the same or separate days. In one embodiment of the presently disclosed subject matter, the active agents are combined and administered in a single dosage form. In another embodiment, the active agents are administered in separate dosage forms (e.g., wherein it is desirable to vary the amount of one but not the other). The single dosage form may include additional active agents for the treatment of the disease state.

Further, TNFR2 modulating agents can be administered alone or in combination with adjuvants that enhance stability of the agents, alone or in combination with one or more therapeutic agents; facilitate administration of pharmaceutical compositions containing them; in certain embodiments, provide increased dissolution or dispersion; increase inhibitory activity; provide adjunct therapy; and the like, including other active ingredients. Advantageously, such combination therapies utilize lower dosages of the conventional therapeutics, thus avoiding possible toxicity and adverse side effects incurred when those agents are used as monotherapies.

The timing of administration of a TNFR2 modulating agent described herein and at least one additional therapeutic agent can be varied so long as the beneficial effects of the combination of these agents are achieved. Accordingly, the phrase “in combination with” refers to the administration of a TNFR2 modulating agent described herein and at least one additional therapeutic agent either simultaneously, sequentially, or a combination thereof. Therefore, a subject administered a combination of a TNFR2 modulating agent described herein and at least one additional therapeutic agent can receive a TNFR2 modulating agent and at least one additional therapeutic agent at the same time (i.e., simultaneously) or at different times (i.e., sequentially, in either order, on the same day or on different days), so long as the effect of the combination of both agents is achieved in the subject.

When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the agent described herein and at least one additional therapeutic agent are administered simultaneously, they can be administered to the subject as separate pharmaceutical compositions, or they can be administered to a subject as a single pharmaceutical composition comprising both agents.

### D. Definitions

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

While the following terms in relation to the present disclosure are believed to be well understood by one of ordinary skill in the art, the following definitions are set forth to facilitate explanation of the presently disclosed subject matter. These definitions are intended to supplement and illustrate, not preclude, the definitions that would be apparent to one of ordinary skill in the art upon review of the present disclosure.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

The term “combination” is used in its broadest sense and means that a subject is administered at least two agents, more particularly a TNFR2 modulating agent and at least one other therapeutic agent. More particularly, the term “in combination” refers to the concomitant administration of two (or more) active agents for the treatment of a, e.g., single disease state.

In general, the “effective amount” of an active agent refers to the amount necessary to elicit the desired biological response. As will be appreciated by those of ordinary skill in this art, the effective amount of an agent may vary depending on such factors as the desired biological endpoint, the agent to be delivered, the makeup of the pharmaceutical composition, the target tissue, and the like.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

As used herein, the term “treating” can include reversing, alleviating, inhibiting the progression of, preventing or reducing the likelihood of the disease, disorder, or condition to which such term applies, or one or more symptoms or manifestations of such disease, disorder or condition. Preventing refers to causing a disease, disorder, condition, or symptom or manifestation of such, or worsening of the severity of such, not to occur. Accordingly, TNFR2 modulating agents of the present disclosure can be administered prophylactically to prevent or reduce the incidence or recurrence of the disease, disorder, or condition.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The specific examples that follow are only intended for the purposes of illustration, and are not to be construed as limiting in any manner to practice methods of the disclosure.

### Example 1

### 1.1 Overview

TNF is a pro-inflammatory cytokine involved in inflammatory conditions and host defense against infection (Reference 51). The potential roles of TNF and its receptors, TNFR1 and TNFR2, in protection against skin infections, however, are not well defined. The present example reveals the roles of TNF, TNFR1 and TNFR2 in promoting protection against skin infections and provides approaches for treating bacterial skin infections.

### 1.2 Results

### 1.2.1 TNF Promotes Host Defense Against S. aureus Skin Infections

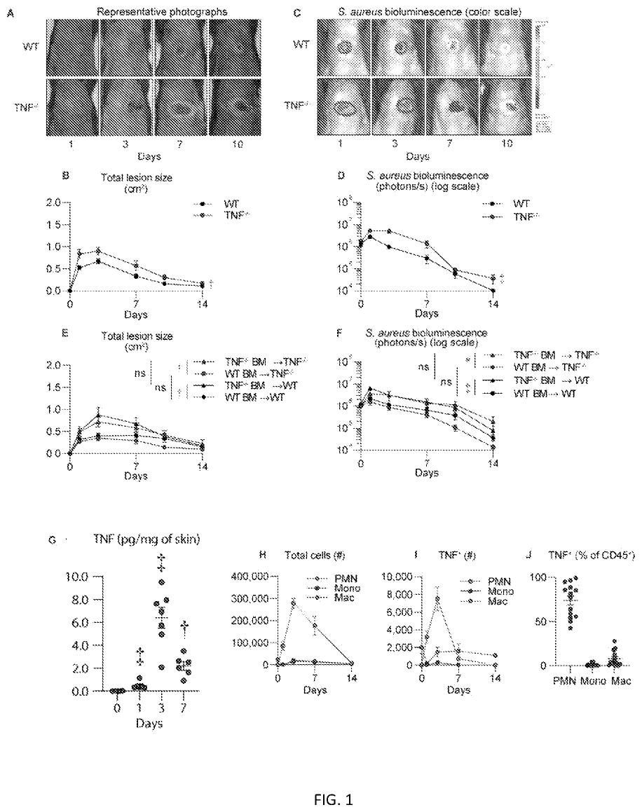

To determine whether tumor necrosis factor (TNF) mediates protection against S. aureus skin infections, an intradermal (i.d.) S. aureus skin infection model was performed in wild-type (WT) and TNF-deficient (TNF^{-/-}) mice (References 16-18). In this model, a bioluminescent CA-MRSA USA300 LAC::lux strain is inoculated intradermally into the dorsal skin of mice. The bacterial burden is monitored noninvasively and longitudinally by in vivo bioluminescence imaging (BLI), which closely approximates ex vivo colony-forming unit (CFU) isolated from the infected skin [R2=0.9996] (Reference 19). We found that TNF^{-/-} mice developed significantly larger skin lesions and higher bacterial burdens than WT mice (FIG. 1A-D), suggesting that TNF contributed to host defense against the S. aureus skin infection.

### 1.2.2 Bone Marrow Cell-Derived TNF is Critical for Host Defense Against S. aureus Skin Infections

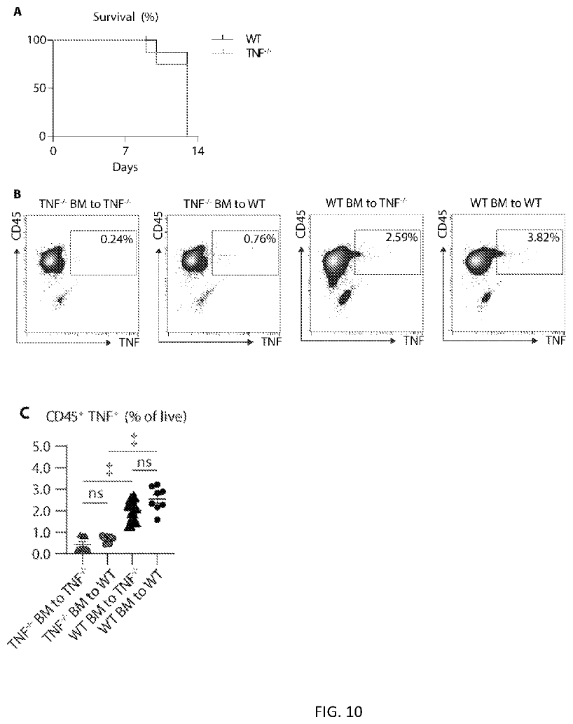

TNF is expressed by immune cells, including neutrophils, monocytes, macrophages, and T cells, as well as non-immune cells, such as keratinocytes and endothelial cells (References 20-24). To determine the differential contribution of immune and non-immune cells to TNF-mediated host defense, we employed a bone marrow (BM) reconstitution experiment in which lethally irradiated WT and TNF^{-/-} mice were reconstituted with BM cells from WT or TNF^{-/-} mice to generate four groups: (1) TNF^{-/-} mice reconstituted with TNF^{-/-} BM (TNF^{-/-} BM→TNF^{-/-}), (2) TNF^{-/-} mice reconstituted with WT BM (WT BM→TNF^{-/-}), (3) WT mice reconstituted with TNF^{-/-} BM (TNF^{-/-} BM→WT), and (4) WT mice reconstituted with WT BM (WT BM→WT). We confirmed lethal irradiation by measuring survival without BM reconstitution (FIG. 10A) and BM reconstitution was confirmed by evaluating TNF-producing cells in circulation by flow cytometry (FACS) analysis (FIG. 10B and FIG. 10C). At 8 weeks post-reconstitution, we intradermally inoculated the BM reconstituted mice with S. aureus and evaluated the skin lesion sizes and bacterial burdens. We found that mice that received BM cells from TNF^{-/-} mice (TNF^{-/-} BM→TNF^{-/-} and TNF^{-/-} BM→WT) had marked skin lesions and bacterial burdens, which were significantly reduced in mice that received BM from WT mice (WT BM→TNF^{-/-} and WT BM→WT) (FIG. 1E and FIG. 1F). Moreover, there were no significant differences in lesion sizes or bacterial burden between WT or TNF^{-/-} mice that received BM cells from WT mice or between WT or TNF^{-/-} mice that received BM cells from TNF^{-/-} mice. These results indicated that TNF originating from BM-derived immune cells, but not skin resident cells, was important for immunity against S. aureus skin infections. These results indicated that TNF originating from BM-derived immune cells was important for immunity against S. aureus skin infections.

### 1.2.3 Neutrophils are a Predominant Source of TNF During S. aureus Skin Infections

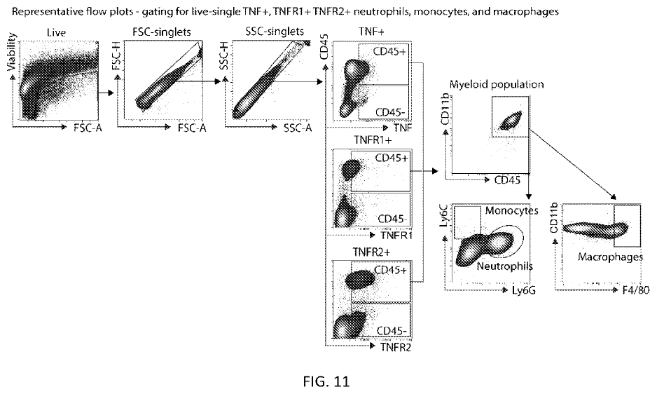

We next set out to determine the kinetics of TNF expression during the S. aureus skin infection to better understand which BM-derived immune cells contributed to TNF production. At baseline, WT mice had negligible TNF levels in the skin, which increased upon S. aureus infection on day 1 post-infection, peaked on day 3 post-infection, and subsided by day 7 post-infection (FIG. 1G). In the serum, WT mice had negligible TNF levels at baseline that did not significantly change upon S. aureus infection (data not shown). Since we observed increased TNF production in the skin as early as day 1 post-infection, without wishing to be bound to any one particular theory, it was thought that innate immune cells were a source of TNF during the S. aureus skin infection. We first quantified the myeloid cells (e.g., neutrophils, monocytes, and macrophages) in the skin during the S. aureus infection and found a notable peak in neutrophils on day 3 post-infection, whereas monocytes and macrophages were lower in comparison (FIG. 1H). To identify whether myeloid cells were a source of TNF production, we performed flow cytometric analysis for intracellular TNF (see gating strategy in FIG. 11) and discovered that neutrophils were a predominant source of TNF among BM-derived immune cells (CD45+) in the skin on day 3 post-infection (FIG. 1I, FIG. 1J). To a lesser extent, there were TNF-producing macrophages in the skin throughout the infection, whereas there were negligible numbers of TNF-producing monocytes.

### 1.2.4 TNFR1 and TNFR2 Signaling are Both Critical for Host Defense Against S. aureus Skin Infections

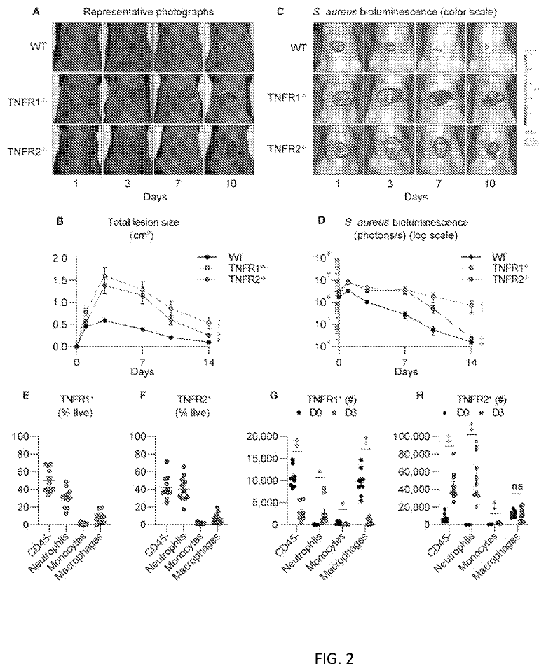

TNF signals through TNFR1 and TNFR2, which have different downstream signaling pathways that dictate various biological outcomes, including inflammatory and host defense responses (References 5, 8). To determine the relative contribution of TNFR1 and TNFR2 to host defense against S. aureus skin infections, we performed our i.d. S. aureus skin infection model in WT, TNFR1^{-/-} deficient (TNFR1^{-/-}), and TNFR2-deficient (TNFR2^{-/-}) mice. We found that TNFR1^{-/-} and TNFR2^{-/-} mice developed significantly increased skin lesions and bacterial burdens compared to WT mice (FIG. 2A-FIG. 2D). These data indicated that TNFR1 and TNFR2 were both important for TNF-mediated host defense against S. aureus skin infections.

### 1.2.5 Neutrophils are a Predominant TNFR1- and TNFR2-Expressing Cell Type During S. aureus Skin Infection

Since both TNFR1 and TNFR2 were crucial for host defense, we next elucidated which cell types respond to TNF via TNFR1 and TNFR2 during the S. aureus skin infection. Therefore, we performed FACS analysis on single-cell suspensions from day 3 infected skin (see gating strategy in FIG. 11) since this corresponded to peak TNF expression during the skin infection (FIG. 1G). By percentage of live cells, we discovered that skin resident cells (CD45⁻) and neutrophils were the predominant TNFR1- and TNFR2-expressing cells in the day 3 infected skin, whereas macrophages and monocytes accounted for lesser amounts of the TNFR1- and TNFR2-expressing cells during the infection (FIG. 2E-FIG. 2F). To determine whether TNFR expression was induced as a response to S. aureus infection, we compared TNFR1 and TNFR2 expression by FACS analysis from single-cell suspensions between uninfected skin (day 0) and day 3 infected skin. Interestingly, there was a marked reduction in TNFR1-expressing skin resident cells, monocytes, and macrophages, whereas there was a significant increase in TNFR1-expressing neutrophils in the day 3 infected skin compared to uninfected skin (FIG. 2G). In contrast, TNFR2-expressing skin resident cells, neutrophils, and monocytes were markedly increased, whereas macrophages had no change in the day 3 infected skin compared to uninfected skin (FIG. 2H). Collectively, populations of TNFR1- and TNFR2-expressing neutrophils were significantly increased in the S. aureus infected skin.

### 1.2.6 Neutrophil-Intrinsic TNFR1 and TNFR2 Signaling Promotes Protection Against S. aureus Skin Infections

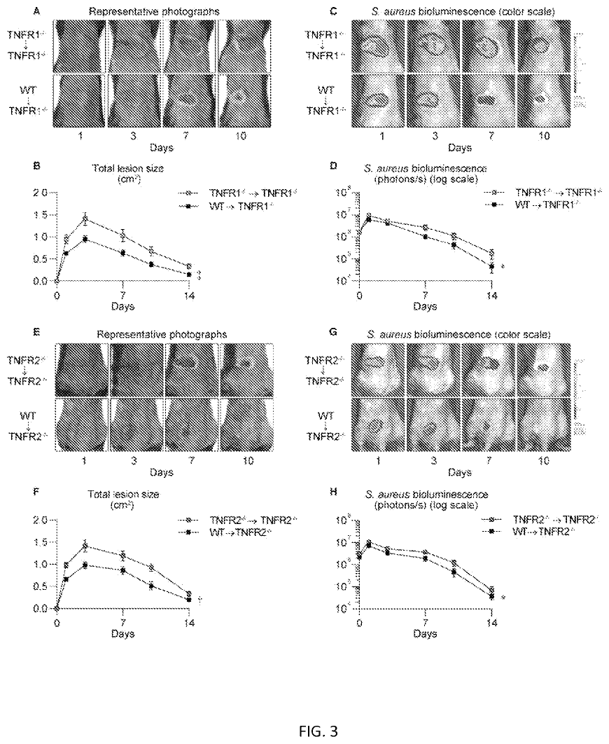

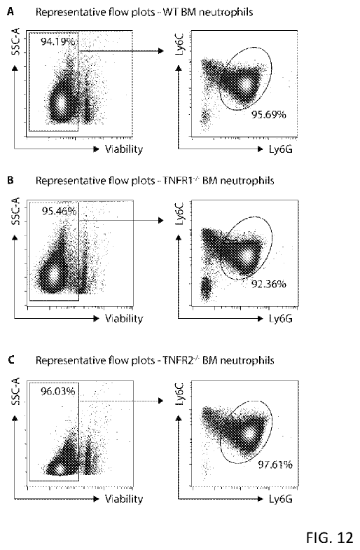

Neutrophil recruitment and abscess formation are critical for the resolution of S. aureus skin infections (References 25-28). Since there were markedly increased TNFR1- and TNFR2-expressing neutrophils in the infected skin, without wishing to be bound to any one particular theory, it was thought that neutrophil-intrinsic TNFR signaling was crucial for protection against S. aureus skin infections. Therefore, we performed adoptive transfer experiments whereby neutrophils purified from the bone marrow of WT or TNFR1^{-/-} mice were adoptively transferred via retro-orbital intravenous (i.v.) injection into TNFR1^{-/-} mice 2 hours prior to S. aureus skin infection. In additional experiments, neutrophils from WT or TNFR2^{-/-} mice were adoptively transferred into TNFR2^{-/-} mice. The viability (>94%) and purity (>92%) of the neutrophils were confirmed before proceeding with the adoptive transfer experiments (FIG. 12A, FIG. 12B and FIG. 12C). Adoptive transfer of WT neutrophils into TNFR1^{-/-} mice (WT→TNFR1^{-/-}) led to significantly reduced lesion sizes and bacterial burden compared to adoptive transfer of TNFR1^{-/-} neutrophils into TNFR1^{-/-} mice (TNFR1^{-/-}→TNFR1^{-/-}) (FIG. 3A-FIG. 3D). Similarly, adoptive transfer of WT neutrophils into TNFR2^{-/-} mice (WT→TNFR2^{-/-}) caused markedly decreased skin lesion sizes and bacterial burden compared to adoptive transfer of TNFR2^{-/-} neutrophils into TNFR2^{-/-} mice (TNFR2^{-/-}→TNFR2^{-/-}) (FIG. 3E-FIG. 3F). Taken together, neutrophil-intrinsic TNFR1 and TNFR2 signaling contributed to immunity against S. aureus skin infections.

### 1.2.7 TNFR1 Signaling Promotes Neutrophilic Abscess Formation in the Skin

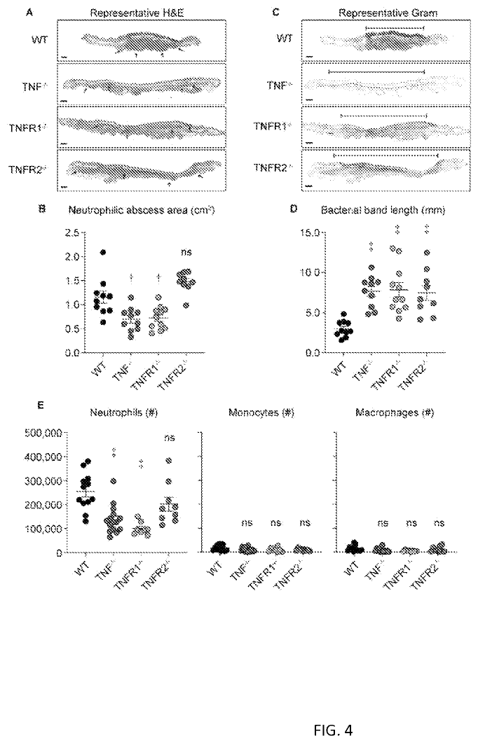

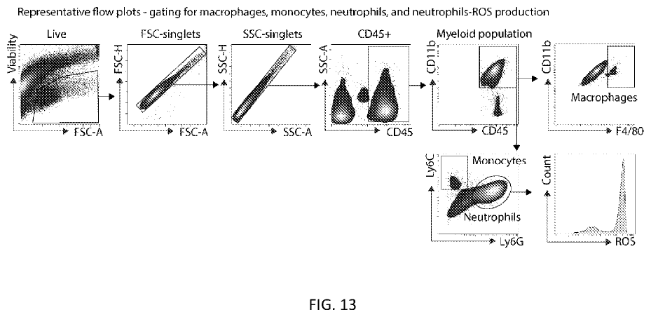

TNF has previously been reported to promote neutrophil recruitment to sites of contact hypersensitivity-induced skin inflammation (Reference 22). Since neutrophilic abscess formation is a hallmark of immunity against S. aureus skin infections (References 25-28), we next asked whether TNF and TNFR signaling affected neutrophil recruitment during the S. aureus skin infection. To this end, we compared neutrophilic abscess formation on day 3 infected skin from H&E-stained skin sections of WT, TNF^{-/-}, TNFR1^{-/-}, and TNFR2^{-/-} mice. Surprisingly, we found that the neutrophilic abscess area (as denoted by the black arrows, FIG. 4A) was abrogated in TNF^{-/-} and TNFR1^{-/-}, but not TNFR2^{-/-} mice compared to WT mice (FIG. 4A, FIG. 4B). In contrast, analysis of Gram-stained skin sections showed that TNF^{-/-}, TNFR1^{-/-}, and TNFR2^{-/-} mice all had significantly increased bacterial band lengths compared to WT mice (FIG. 4C, FIG. 4D). To confirm our results, we performed FACS analysis from day 3 infected skin (see gating strategy in FIG. 13) and found a similar pattern as our neutrophilic abscess area results whereby neutrophil numbers were markedly decreased in the skin of TNF^{-/-} and TNFR1^{-/-}, but not TNFR2^{-/-} mice compared to WT mice (FIG. 4E). Interestingly, these findings were specific to neutrophils, as monocytes and macrophages had comparable levels between WT mice and TNF^{-/-}, TNFR1^{-/-}, and TNFR2^{-/-} mice (FIG. 4E). Collectively, our data suggest that TNFR1 signaling promotes neutrophil trafficking and ensuing neutrophilic abscess formation for optimal host defense against S. aureus skin infections.

### 1.2.8 Neutrophil Extracellular Traps (NETs) Foster Protection Against S. aureus Skin Infections

Neutrophil extracellular traps (NETs) form upon neutrophil exposure to an external stimulus, including TNF and S. aureus (References 29-31), which induces the release of decondensed chromatin decorated with antimicrobial proteins to trap and kill microbes (Reference 32). The role of NETs in host defense against S. aureus skin infections, however, is not entirely clear. To determine if NET formation is protective during S. aureus skin infections, mice deficient in peptidylarginine deiminase 4 (PAD4^{-/-}), which facilitates chromatin decondensation and subsequent NET formation (Reference 33), were intradermally inoculated with S. aureus. We discovered that PAD4^{-/-} mice developed significantly increased skin lesion sizes and bacterial burden relative to WT mice (FIG. 5A-FIG. 5D), which was most pronounced at time points when neutrophils migrated into the skin (FIG. 1H). Importantly, PAD4^{-/-} mice had comparable neutrophilic abscess areas and neutrophil recruitment in the skin compared to WT mice (FIG. 5E-FIG. 5G) despite a larger bacterial band length (FIG. 5H, FIG. 5I). Together, these data suggested that PAD4-dependent NET formation is an important host defense mechanism against S. aureus skin infection.

### 1.2.9 TNFR2 Signaling Promotes NET Formation in the Skin

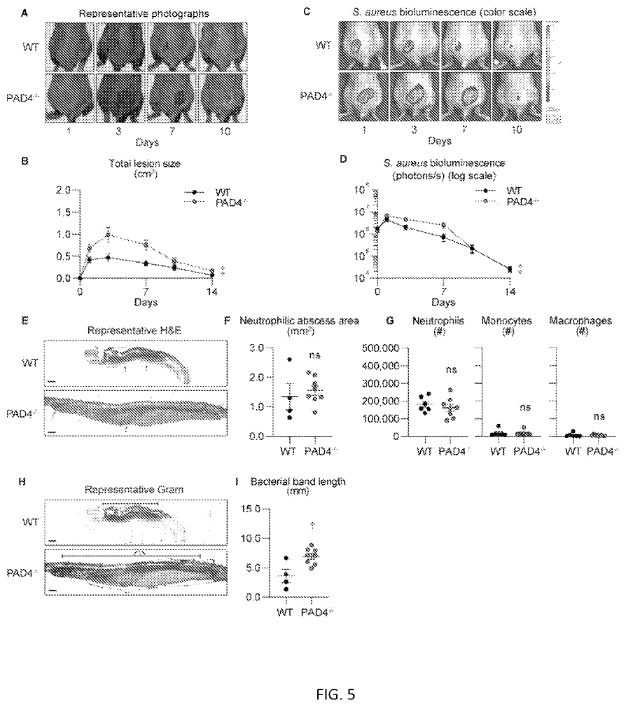

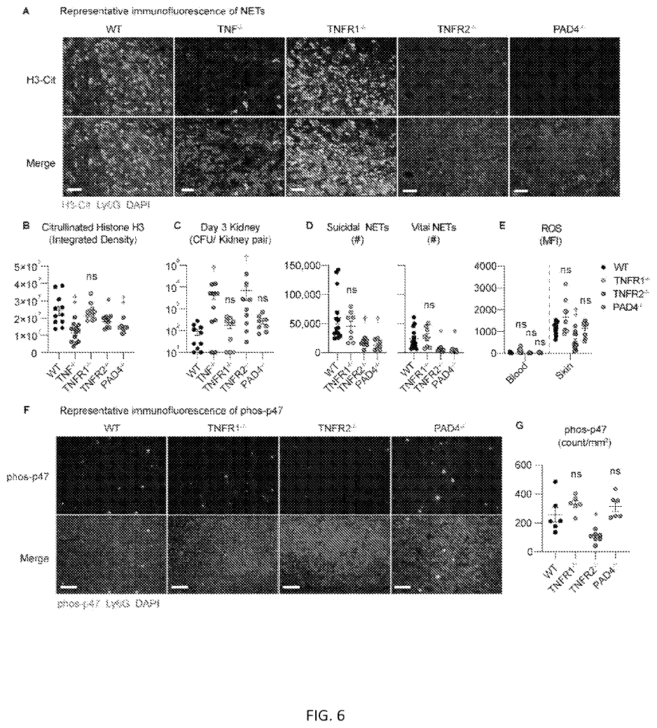

Since TNF facilitates NET formation in vitro (References 29, 34), without wishing to be bound to any one particular theory, it was thought that TNF signaling was important for NET formation during S. aureus skin infection. To determine the role of TNF and the differential contributions of TNFR1 and TNFR2 in NET formation during the S. aureus skin infection, we examined day 3 infected skin sections from WT, TNF^{-/-}, TNFR1^{-/-}, and TNFR2^{-/-} mice for citrullinated histone 3 (H3-Cit) and Ly6G, which are established markers for NET formation (References 34-36). Additionally, we included PAD4^{-/-} mice as a negative control for NET formation. As expected, infected skin from WT mice exhibited robust NET formation as indicated by intense H3-Cit stain in proximity to Ly6G, which was markedly diminished in PAD4^{-/-} mice (FIG. 6A, FIG. 6B). Notably, infected skin from TNF^{-/-} and TNFR2^{-/-}, but not TNFR1^{-/-} mice showed markedly abrogated H3-Cit stain compared to WT mice (FIG. 6A). H3-Cit intensity was quantified by integrated fluorescence intensity, which showed a significant reduction in TNF^{-/-}, TNFR2^{-/-}, and PAD4^{-/-} mice, but not in TNFR1^{-/-} mice compared to WT mice (FIG. 6B). Since NETs immobilize and control spreading of pathogens (References 37, 38), we next asked whether TNF-mediated NET formation prevented bacterial dissemination from the skin to kidneys on day 3 of the infection. Surprisingly, while we saw significantly higher bacterial burdens in the kidneys of TNF^{-/-} and TNFR2^{-/-} mice, TNFR1^{-/-} and PAD4^{-/-} had comparable CFU counts in the kidney compared to WT mice (FIG. 6C). Collectively, these results indicated that TNFR2 signaling promotes NET formation and prevents bacterial dissemination during S. aureus skin infections.

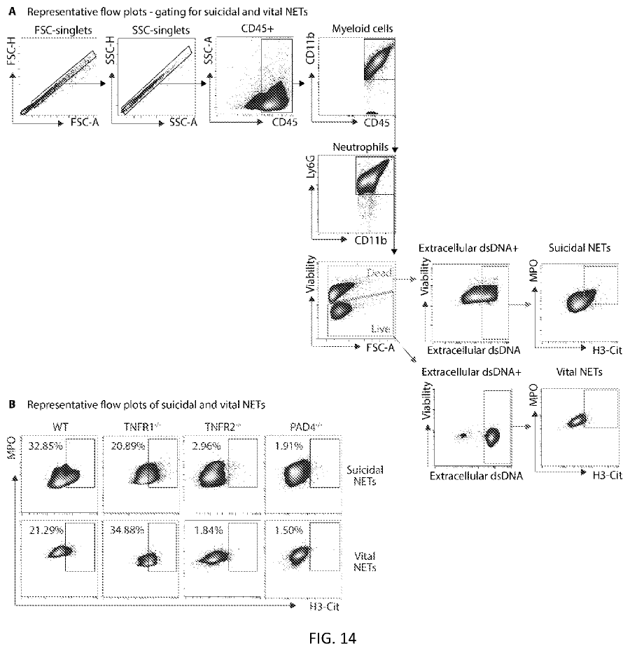

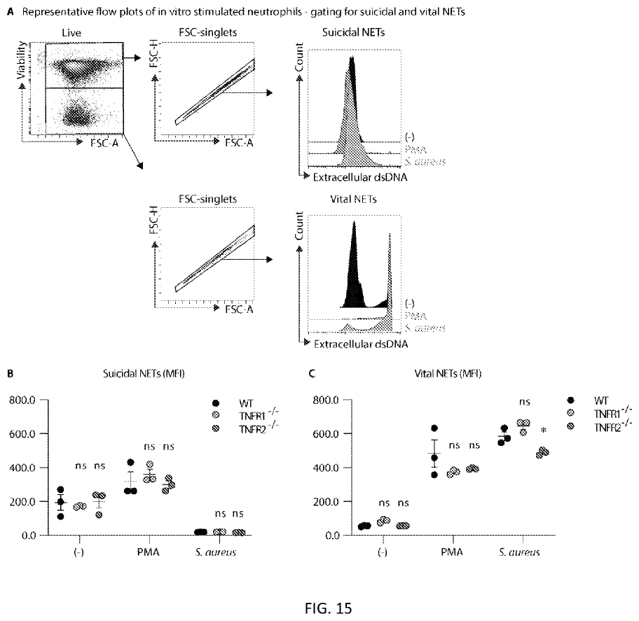

Depending on the type and duration of the external stimuli, neutrophils form either suicidal or vital NETs (Reference 39). Suicidal NET formation involves cell membrane lysis that leads to cell death, whereas vital NET formation relies on the release of decondensed chromatin from vesicles, leaving the cell membrane intact for cell survival (References 40, 41). It is unknown, however, which type of NETs form during S. aureus infections in the skin or how TNF signaling influences suicidal or vital NET formation. To evaluate the levels of suicidal and vital NET formation in the skin, neutrophils from the skin of WT, TNFR1^{-/-}, TNFR2^{-/-}, and PAD4^{-/-} mice were assessed by FACS analysis as previously described (see gating strategy in FIG. 14A) (Reference 42). While suicidal and vital NET formation was comparable between WT and TNFR1^{-/-} mice, both suicidal and vital NET formation were significantly diminished in TNFR2^{-/-} and PAD4^{-/-} mice compared to WT mice (FIG. 6D and FIG. 14B). In vitro stimulation of WT, TNFR1−/−, and TNFR2−/− neutrophils with phorbol myristate acetate (PMA) or live S. aureus induced vital but not suicidal NET formation (FIG. 15A-C). Consistent with our in vivo findings, TNFR2−/− neutrophils co-cultured with S. aureus had significantly reduced vital NET formation compared to WT neutrophils (FIG. 15C). These findings indicated that TNFR2 signaling was crucial for both suicidal and vital NET formation in response to the S. aureus skin infection.

### 1.2.10 TNFR2 Signaling Initiates ROS Generation Via NOX2 Activation in Neutrophils

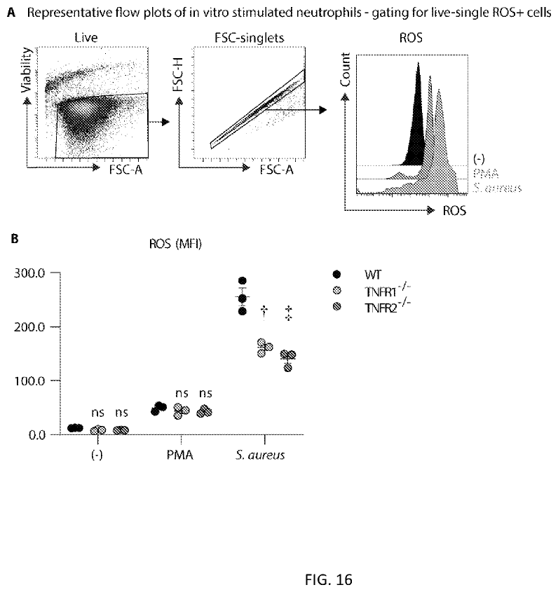

Suicidal NET formation is dependent on the generation of reactive oxygen species (ROS) in neutrophils (References 39, 43). Given our findings that TNFR2 is important for suicidal NET formation, without wishing to be bound to any one particular theory, it was thought that TNFR2 signaling induced ROS production in neutrophils during S. aureus skin infections. Thus, we used FACS analysis to examine the ROS levels in neutrophils from day 3 infected skin and blood of WT, TNFR1^{-/-}, TNFR2^{-/-}, and PAD4^{-/-} mice. Interestingly, ROS production was negligible in circulating neutrophils in all the tested groups, whereas upon infiltration into the infected skin, WT neutrophils had robust ROS production that was markedly decreased in TNFR2^{-/-} neutrophils, but not in TNFR1^{-/-} and PAD4^{-/-} neutrophils (FIG. 6E). In vitro stimulation of neutrophils with live S. aureus induced ROS production (FIG. 16), which was dependent on both TNFR1 and TNFR2 signaling (FIG. 16B).

Neutrophils express NADPH oxidase 2 (NOX2) that enables robust production of ROS for antimicrobial host defense (References 44, 45). NOX2 is a multi-component enzymatic complex that becomes activated upon assembly of cytosolic components (p47ₚₕₒₓ, p67ₚₕₒₓ, p40ₚₕₒₓ, and Rac2) with its transmembrane proteins (p22ₚₕₒₓ and gp91ₚₕₒₓ) (46, 47). Phosphorylation of p47ₚₕₒₓ is critical for the assembly of the cytosolic subunits and mobilization of NOX2 to initiate ROS production (Reference 47). To determine whether ROS production in neutrophils involved NOX2 activation downstream of TNFR2 signaling, we performed immunofluorescent microscopy for phosphorylated p47ₚₕₒₓ (phos-p47) on day 3 infected skin sections from WT, TNFR1^{-/-}, TNFR2^{-/-}, and PAD4^{-/-} mice. Consistent with our ROS findings, WT infected skin had abundant phos-p47 puncta in the neutrophilic abscess that was significantly reduced in TNFR2^{-/-} skin, but not in TNFR1^{-/-} and PAD4^{-/-} skin (FIG. 6F, FIG. 6G). These data indicated that TNFR2 signaling induced ROS production via NOX2 activation in neutrophils during S. aureus skin infections.

### 1.2.11 A TNFR2 Agonist has Efficacy Against S. aureus Skin Infections in Mice

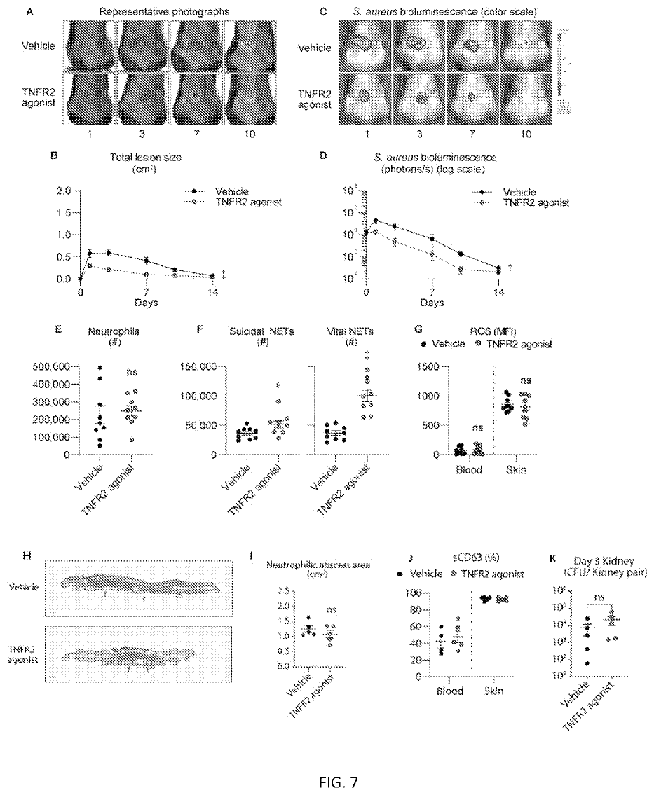

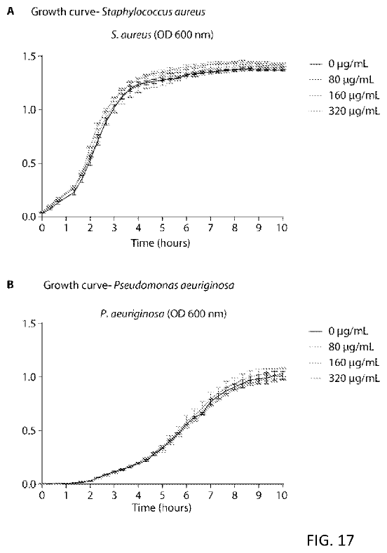

Since TNF inhibitors promote S. aureus skin colonization and infection in patients (References 14, 15), and TNFR2 signaling drives neutrophil NET formation and NOX2-mediated ROS production, we postulated that TNFR2 agonism would enhance host defense against S. aureus skin infections via increased neutrophil activation. To this end, we therapeutically treated WT mice with a single intraperitoneal (i.p.) dose of a TNFR2 agonist (Reference 48) or vehicle 4 hours following the S. aureus intradermal inoculation. The TNFR2 agonist treatment resulted in markedly reduced skin lesion sizes (FIG. 7A, FIG. 7B) and bacterial burdens (FIG. 7C, FIG. 7D) as compared to vehicle-treated mice. The efficacy of the TNFR2 agonist against the S. aureus skin infection was not due to direct anti-staphylococcal activity, as there were no differences in the growth curves of S. aureus incubated with varying concentrations of the TNFR2 agonist (80, 160, and 320 μg/mL) and vehicle control (FIG. 17A). To note, the 160 μg/mL TNFR2 agonist concentration corresponded to the equivalent dose administered to the mice.

To evaluate whether the TNFR2 agonist altered neutrophil recruitment and/or function, we measured neutrophil levels, suicidal and vital NET formation, and neutrophil ROS production following TNFR2 agonist or vehicle treatment in day 3 infected skin. While there were no significant differences in the neutrophilic abscess area (FIG. 7H and FIG. 7I) and the number of neutrophils in the skin (FIG. 7E), we observed a marked increase in suicidal and vital NET formation in TNFR2 agonist-treated mice compared to vehicle-treated mice (FIG. 7F). Despite an increase in suicidal NET formation, we found no differences in the ROS production (FIG. 7G) and primary degranulation (FIG. 7J) between TNFR2 agonist and vehicle-treated neutrophils in circulation and skin. Finally, to determine if TNFR2 agonist could further block systemic dissemination of skin infection, we compared the bacterial burden in the kidneys at day 3 between vehicle-treated and TNFR2 agonist-treated mice. The CFU counts per kidney pair were comparable between vehicle- and TNFR2 agonist-treated mice (FIG. 7K). Taken together, TNFR2 agonist treatment promoted host defense against S. aureus skin infections by a mechanism that involved increased suicidal and vital NET formation.

### 1.2.12 TNFR2 Agonist Treatment has Efficacy Against Pseudomonas aeruginosa Skin Infections in Mice

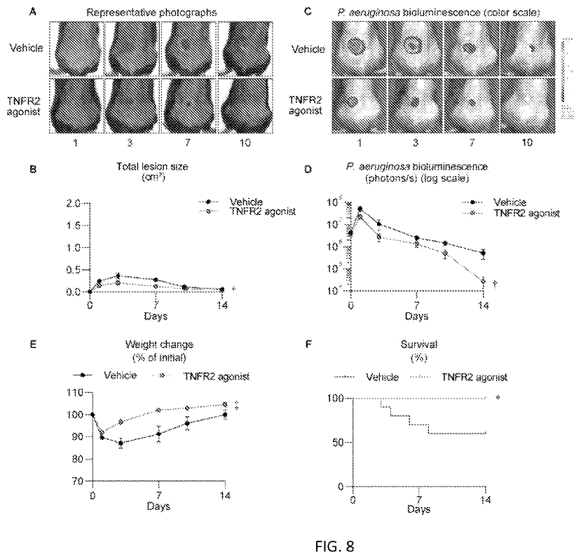

Given the efficacy of the TNFR2 agonist against S. aureus, a Gram-positive skin pathogen, we next examined whether the antimicrobial efficacy extended to the Gram-negative skin pathogen, Pseudomonas aeruginosa (References 4, 49). Therefore, we performed our intradermal infection model with a bioluminescent P. aeruginosa strain [Xen41 (Reference 50)] in WT mice. A single dose of the TNFR2 agonist or vehicle was administered i.p. 4 hours post-infection, and lesion sizes and in vivo BLI were longitudinally measured over 14 days. Similar to the S. aureus skin infection, TNFR2 agonist-treated mice had substantially decreased skin lesion sizes (FIG. 8A, FIG. 8B) and reduced bacterial burden compared to vehicle-treated mice (FIG. 8C, FIG. 8D). Moreover, the P. aeruginosa skin infection caused marked reductions in weight and survival in the vehicle-treated mice, which were significantly improved in the TNFR2 agonist-treated mice (FIG. 8E, FIG. 8F). The efficacy of the TNFR2 agonist against the P. Aeruginosa skin infection was not due to direct anti-bacterial activity, as there were no differences in the growth curves of P. aeruginosa incubated with varying concentrations of the TNFR2 agonist (80, 160, and 320 μg/mL) and vehicle control (FIG. 17B). These results indicated that the TNFR2 agonist had therapeutic efficacy against P. aeruginosa skin infections.

### 1.2.13 TNF is Dispensable for the Expression of Neutrophil Chemokines, Proinflammatory Cytokines, and Antimicrobial Peptides

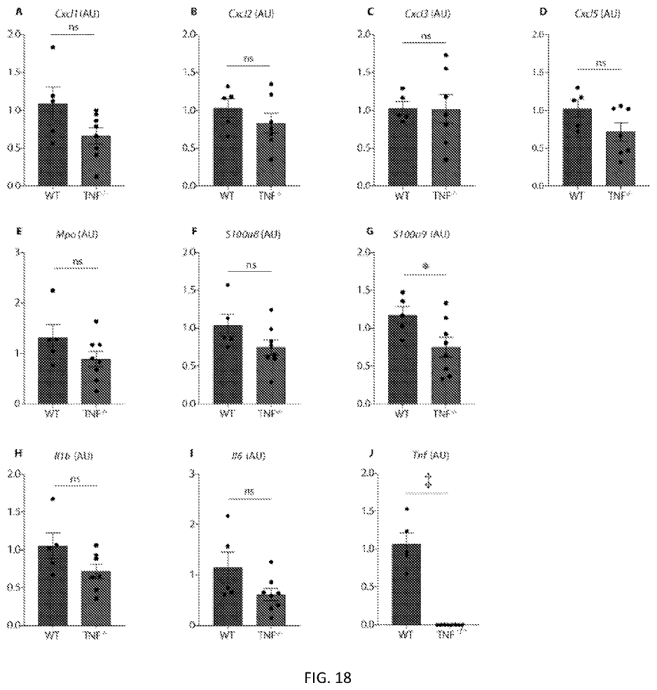

TNF signaling has previously been shown to sustain the expression of inflammatory factors by downstream activation of NFκB signaling that can contribute to the progression of inflammatory disease pathogenesis in dysregulation (Reference 51). Moreover, we observed there was abrogated neutrophilic abscess formation and recruitment to the skin in TNF-mice compared to WT mice (FIG. 4A-FIG. 4E). Therefore, to determine whether TNF contributed to the expression of neutrophil chemokines, day 3 skin biopsies were evaluated for mRNA levels of Cxcl1, Cxcl2, Cxcl3, and Cxcl5 (FIG. 18A-FIG. 18D). Surprisingly, there was no difference between mRNA expression of neutrophil chemokines in TNF^{-/-} mice and WT mice.

TNF with IL-17A has also been shown to synergistically induce the expression of AMPs such as human β-defensin 2 (hBD2) in keratinocytes through indirect activation of NFκB (Reference 97). Furthermore, multiple studies have demonstrated the ability of neutrophils to synthesize cytokines and functional proteins de novo upon stimulation (References 98, 99), but whether TNF regulates the expression of cytokines and AMPs implicated in immunity against S. aureus skin infection is incompletely understood. Therefore, we compared mRNA transcript expression of myeloperoxidase (Mpo), calprotectins (S100a8 and S100a9), Il1b, Il6, and TNF (FIG. 18E-FIG. 18J). TNF^{-/-} mice only showed markedly decreased expression of S100a9 (FIG. 18G) and showed a similar level of expression of Mpo, S100a8, Il1b, and Il6 as WT mice (FIG. 18E, FIG. 18F, FIG. 18H, and FIG. 18I). As expected, Tnf was not detected in TNF^{-/-} mice (FIG. 18J).

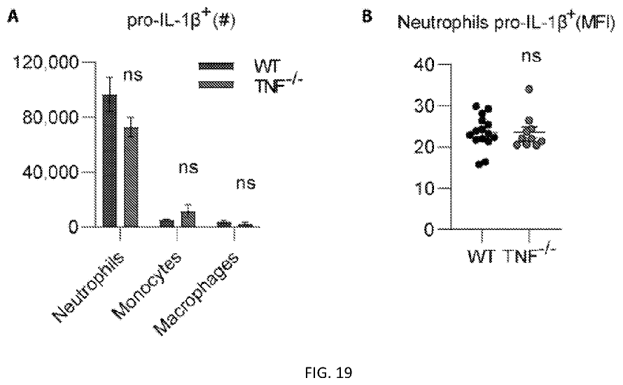

It is well established that inflammasome-dependent IL-1β production, specifically neutrophil-derived IL-1β, is critical to promote sufficient neutrophil responses during S. aureus skin infection (References 26, 27). Furthermore, TNF signaling pathway has been shown to be functionally dependent on IL-1β for optimal antibacterial responses (Reference 100). To check for IL-1β protein expression differences, we performed flow cytometry on day 3 infected skin from WT and TNF^{-/-} mice (FIG. 19A, FIG. 19B). There was no significant difference in the total number of pro-IL-1β+ neutrophils, monocytes, nor macrophages (FIG. 19A). Furthermore, the intensity of pro-IL-1β expression in neutrophils was comparable between WT and TNF^{-/-} mice (FIG. 19B). Together, we confirmed at both mRNA and protein levels that TNF does not promote IL-1β-mediated immune responses.

### 1.2.14 TNF is Critical for IL-17 Expression by T Cells During S. aureus Infection

Clonal population of Vγ6+Vδ4+ T cells mediate IL-17 responses that are critical to immunity against S. aureus skin infection (References 16, 17, 25). Since TNF can prime monocytes and dendritic cells to facilitate Th17 differentiation (Reference 101), we examined if TNF was acting upstream of T helper cell differentiation to promote IL-17 production during the infection. On day 7 of infection, we quantified the total number of IL-17A+γδ+ and CD4+ T cell in the infected skin and draining lymph nodes (dLNs) of WT and TNF^{-/-} mice by flow cytometry analysis (FIG. 20A-FIG. 20F). In the skin, while there was no significant difference in the total number of γδ+ and CD4+ T cell (FIG. 20A), there was significantly less IL-17A+γδ+ and CD4+ T cells in TNF^{-/-} mice compared to WT mice (FIG. 20B and FIG. 20C). In the dLNs, while there were similar number of γδ+ T cells, TNF^{-/-} mice had significantly more CD4+ T cells than WT mice (FIG. 20D). Moreover, the number of CD4+ T cells was about 2 to 3 logs higher than the number of γδ+ T cells in the dLNs (FIG. 20D). Interestingly, TNF^{-/-} mice only had significantly lower IL-17A+γδ+ T cells and comparable number of IL-17A+CD4+ T cells to WT mice (FIG. 20E and FIG. 20F). Together, our data indicate that TNF regulates the expression of IL-17 by both γδ+ and CD4+ T cell in the skin and the dLNs in response to S. aureus skin infection.

### 1.2.15 Adoptive Transfer of WT Neutrophils does not Rescue the Immune Defect in TNF^{-/-} Mice

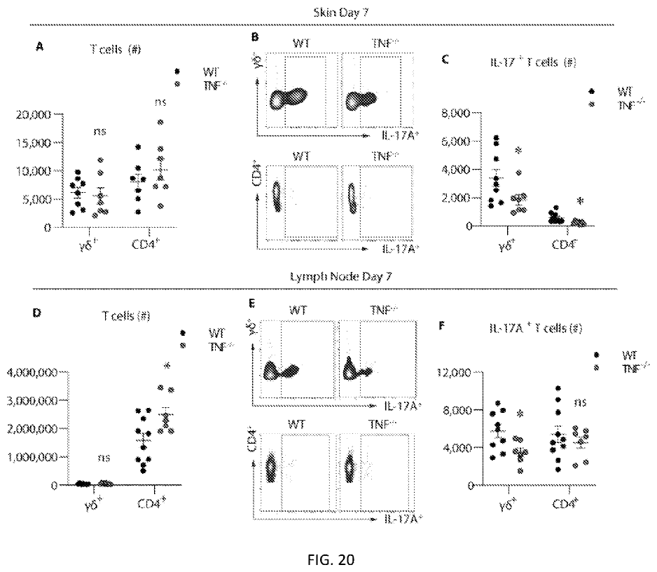

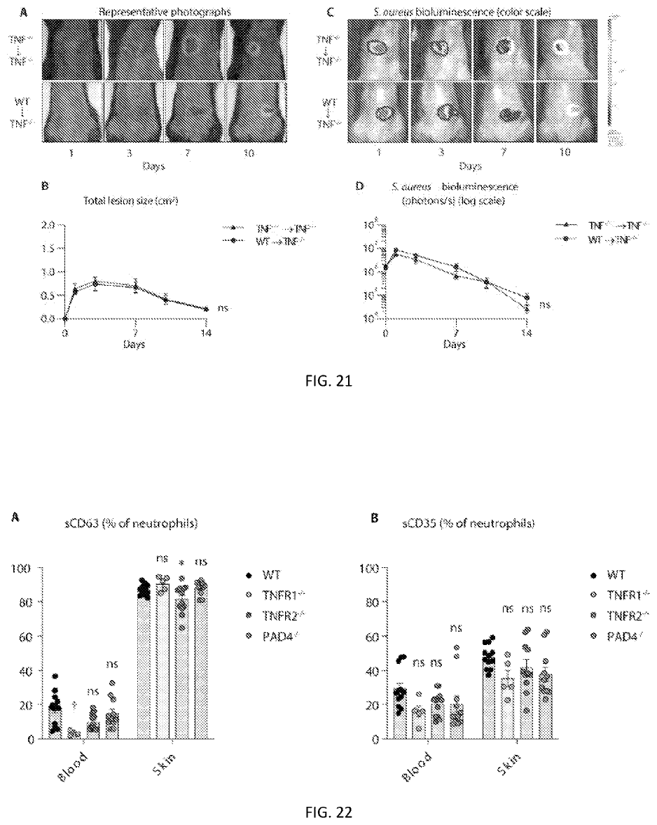

Since neutrophils were made up ~75% of TNF-expressing cells in the skin on day 3 post-infection (FIG. 1J), we hypothesized neutrophil-derived TNF was predominantly promoting host protection against S. aureus. We adoptively transferred neutrophils purified from the bone marrow of WT or TNF^{-/-} mice through retro-orbital intravenous (i.v.) injection into TNF^{-/-} mice 2 hours before infection. Surprisingly, there were no differences in lesion size and bacterial burden between adoptive transfer of WT neutrophils into TNF^{-/-} (WT→TNF^{-/-}) and adoptive transfer of TNF^{-/-} neutrophils into TNF^{-/-} mice (FIG. 21A-FIG. 21D), suggesting a more complicated cellular and molecular mechanism that involves neutrophils to induce host-protective concentrations of TNF in the skin after S. aureus infection.

### 1.2.16 TNFR2 is Critical for Azurophilic Degranulation of Neutrophils in the Skin

A major immune response by neutrophils is the exocytosis of granules packaged with proteases and antimicrobial proteins (Reference 102). In vitro stimulation of neutrophils with TNF has previously been shown to stimulate secretory and gelatinase degranulation while priming cells to go through specific and azurophilic degranulation upon secondary stimuli (Reference 103). Thus, we evaluated the ability of TNFR1 and TNFR2 to induce azurophilic (CD63+) and secretory degranulation (CD35+) in neutrophils through flow cytometry analysis. In the blood, less than ~20% of neutrophils from WT, TNFR^{-/-}, and TNFR2^{-/-} and PAD4^{-/-} mice went through azurophilic degranulation (FIG. 22A). Upon migration to the skin, the majority (~90%) of WT, TNFR1^{-/-}, and PAD4^{-/-} neutrophils went through azurophilic degranulation (FIG. 22A). While there was an increase from the blood to the skin, a significantly lower percentage of TNFR2^{-/-} neutrophils went through azurophilic degranulation compared to WT neutrophils in the skin (FIG. 22A). Unlike azurophilic degranulation, we observed no significant changes in secretory degranulation in the blood nor the skin between WT, TNFR1^{-/-}, TNFR2^{-/-}, and PAD4^{-/-} mice (FIG. 22B). Our data suggested that TNFR2 mainly promoted azurophilic degranulation of neutrophils in the skin as a host defense response to S. aureus skin infection.

### 1.2.17 TNFR2 Differentially Regulates Gene and Protein Expression During S. aureus Skin Infection

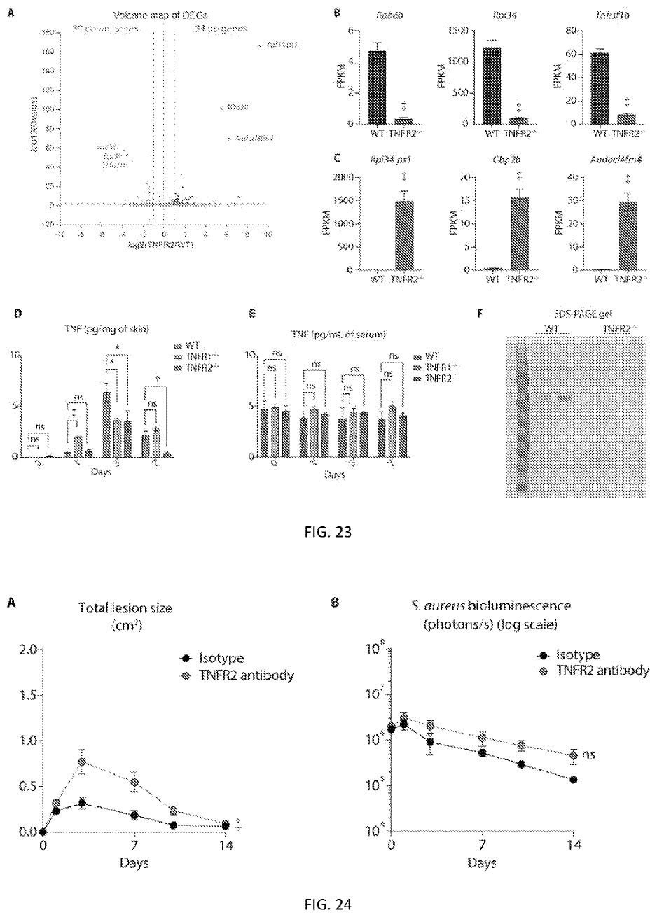

TNFR2 downstream signaling engages the assembly of complex I that is responsible for the expression of cell survival and proliferation factors, and proinflammatory cytokines (Reference 5). Therefore, to determine whether TNFR2 contributed to the global transcriptional regulation of genes important for host protective immune responses, we performed RNA sequencing on day 3 skin biopsies of WT and TNFR2^{-/-} skin (FIG. 23A-FIG. 23F). Whole transcriptomic analysis revealed 30 down-regulated genes and 34 up-regulated genes in day 3 infected TNFR2^{-/-} skin in comparison to WT skin (FIG. 23A). Surprisingly, the most significantly affected transcripts were not cytokines, antimicrobial peptides, nor chemokines. Top 3 most significantly downregulated and upregulated genes were Rab6b, Rpl34, and Tnfrsf1b (as expected in TNFR2^{-/-} mice) (FIG. 23B) and Rpl34-ps1, Gbp2b, and Aadacl4fm4 (FIG. 23C), respectively.

Since TNFR2 had limited transcriptional regulation of genes, but dramatic effects on neutrophil cellular function, we hypothesized that TNFR signaling mediated post-transcriptional modifications. Previously, Etemadi et al. have shown TNF stimulation of macrophages amplifies the production of TNF in a TRAF2-dependent manner (Reference 104). Therefore, we determined the effect of TNFR1 and TNFR2, both of which signal via TRAF2 downstream of receptor activation, on TNF production in the skin. We discovered that TNFR1^{-/-} mice had a transient defect in TNF production in the skin on 3 d.p.i. whereas TNFR2^{-/-} mice had a sustained defect in TNF production from 3 d.p.i. to 7 d.p.i. when compared to WT mice (FIG. 23D). In contrast, there were comparable levels of TNF in the sera between WT, TNFR1^{-/-}, and TNFR2^{-/-} mice, which was stable throughout the course of the infection (FIG. 23E). Then, we tested if the sustained defect of TNF production in TNFR2^{-/-} mice was due to decreased global translational mechanism. We isolated 1.0×10⁴ circulating neutrophils from the blood of WT and TNFR2^{-/-} mice and compared the total protein quantity on a SDS-PAGE gel where we observed noticeably less protein in TNFR2^{-/-} mice compared to WT mice (FIG. 23F).

Finally, we utilized a monoclonal antibody to block TNFR2 signaling in vivo to confirm the immune defects observed in TNFR2^{-/-} mice were not due to developmental defects. WT mice treated with anti-TNFR2 antibody developed significantly larger skin lesions (FIG. 24A) and showed significantly higher BLI (FIG. 24B) compared to WT mice treated with control antibody (isotype). Together, these data indicated that TNFR2 signaling was important for mediating TNF-stimulated cellular and molecular mechanisms of host defense against S. aureus skin infections.

### 1.2.18 TNFR2 Signaling Promotes Granularity in Neutrophils

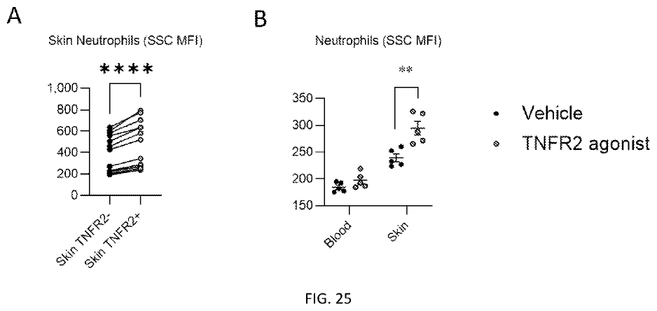

We have found that TNFR2 signaling promotes host defense via neutrophil activation. Since granule formation and release is an important aspect of neutrophil-mediated host defense, it was thought that TNFR2 signaling in neutrophils triggered granule formation. To this end, S. aureus USA300 LAC::lux (3×10⁷ CFU) intradermal skin infections were performed on WT mice and neutrophils from day 3 infected skin examined by flow cytometry. We discovered that TNFR2-expressing neutrophils had significantly increased granularity as measured by SSC MFI compared to non-TNFR2-expressing neutrophils (FIG. 25A). Additionally, we therapeutically treated WT mice with a single intraperitoneal dose of a TNFR2 agonist [8 mg/kg, intraperitoneally (i.p.)] or vehicle (sterile PBS) 4 hours following the S. aureus intradermal inoculation. The TNFR2 agonist treatment resulted in markedly increased granularity in skin but not circulating blood neutrophils on day 3 post-infection compared to vehicle controls (FIG. 25B). Collectively, our findings indicated that TNFR2 signaling promotes granule formation in neutrophils, which may be an important component of the efficacy of the TNFR2 agonist against bacterial skin infections.

### 1.3 Discussion

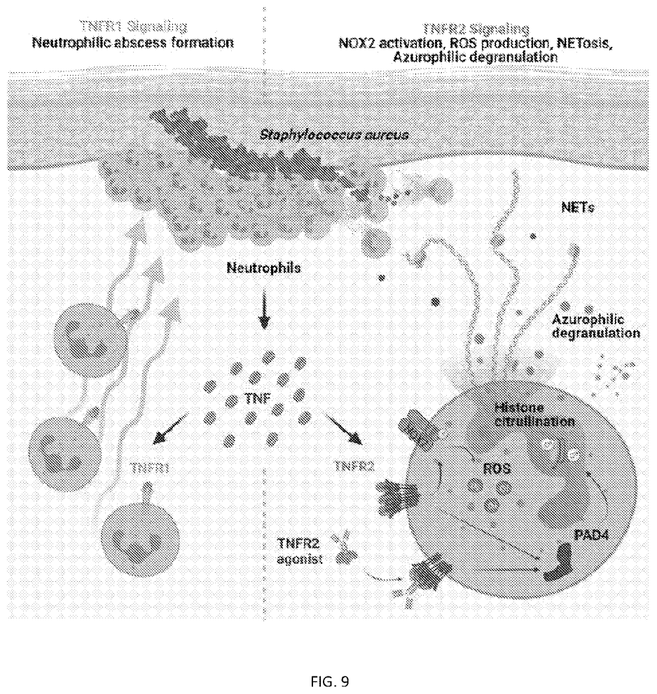

TNF is a pleiotropic cytokine involved in inflammatory conditions and host defense against infection (Reference 51). The role of TNF in the protection against S. aureus skin infections, however, is not well defined. Herein, we discovered that TNF has a crucial host defense role against S. aureus in the skin, with neutrophils as both a major TNF-producing cell and a predominant TNFR1- and TNFR2-expressing cell during the S. aureus skin infection (FIG. 9). Furthermore, TNFR1 and TNFR2 had critical and non-redundant roles in host defense that included neutrophil recruitment and abscess formation, and neutrophil activation (e.g., ROS production and NET formation), respectively. Importantly, targeting TNFR2 with a TNFR2 agonist enhanced NET formation and protected against clinically relevant Gram-positive and Gram-negative skin pathogens (e.g., S. aureus and P. aeruginosa). Collectively, these findings provide new and important insights into TNF biology and function in skin immunity.

First, we found that TNF had an important host defense role against S. aureus in the skin. This finding provides a potential explanation for the increased susceptibility of patients on TNF inhibitors to develop S. aureus skin infections (References 14, 52) and aligns with reports by our group and others that TNF responses are protective against S. aureus at other infection sites, including orthopedic implant-associated infections (Reference 53), bacteremia (Reference 54), and brain abscess infections (References 55, 56).

One of ordinary skill in the art at the time of this filing would have expected TNFR2 signaling to induce immunoregulatory and anti-inflammatory responses, primarily through Tregs. Thus, it was unexpected that TNFR2 promoted host defense rather than immunosuppression in this context.

Additionally, we discovered that BM cell-derived TNF promoted bacterial clearance, with neutrophils as a major producer of TNF during the S. aureus infection in the skin. Similarly, neutrophils produced TNF in response to Mycobacterium tuberculosis infection (Reference 57) and macrophage/neutrophil-derived TNF was critical for protection and survival against a systemic Listeria monocytogenes infection (Reference 58), indicating that myeloid cell-derived TNF is a relevant immune response against bacterial infections.

We observed that TNFR1 and TNFR2 had dynamic expression patterns during the S. aureus skin infection. TNFR1 expression was primarily localized to non-immune cells (i.e., CD45⁻ cells) and macrophages in naïve skin that was reduced upon the S. aureus skin infection. In contrast, there was an increase in TNFR2-expressing non-immune cells and neutrophils during the S. aureus skin infection. This observation was surprising, as the role of TNFR2 in non-immune cells in the skin (e.g., keratinocytes) is largely unexplored (5, 8, 11). Although TNFR2 expression in the epidermis was reported in the context of skin tumorigenesis (Reference 59), the contribution of TNFR2 signaling in keratinocytes or other non-immune cells to the resolution of S. aureus skin infections will be interrogated in our future work.

We also discovered that TNFR1 and TNFR2 had non-redundant contributions to immunity against S. aureus skin infections, which involved neutrophil-intrinsic signaling. For instance, we observed that TNFR1 signaling impacted neutrophil recruitment and neutrophilic abscess formation at the site of the S. aureus skin infection. A potential explanation for this finding is that TNFR1 engagement directly primed neutrophils to extravasate into the skin, as was previously reported in a mouse model of contact hypersensitivity (Reference 22). In contrast, Miller et. al. showed no immune impairment in TNFR1-deficient mice during a S. aureus skin infection (Reference 60). This discrepancy may be due to differences in the S. aureus strains (e.g., MRSA versus MSSA), which include a greater ability of MRSA to intracellularly survive and replicate in macrophages (Reference 61), increased hemolytic activity (Reference 62), and elevated expression of extracellular enzymes (e.g., aureolysin, serine proteases, thermonuclease, and lipase) and toxins (e.g., haemolysins) that manipulate host innate immunity compared to MSSA (Reference 63). This may be particularly relevant as S. aureus protein A (SpA) binds and activates TNFR1 (Reference 64), including on human neutrophils to induce IL-8, TNF, and MIP-1a, expression (Reference 65). Furthermore, SpA promotes TNFR1 shedding to dampen inflammatory outcomes (Reference 66, 67), which may explain the reduced TNFR1 expression on non-immune cells and macrophages during the S. aureus skin infection compared to naïve skin.

We discovered that TNFR2 promoted S. aureus clearance in the skin, in a mechanism that involved neutrophil activation. This was unexpected since TNFR2 is associated with immunosuppressive functions in other immune cells (References 68, 69). For example, TNFR2 drives Treg differentiation, expansion, and anti-inflammatory responses in models of autoimmunity (References 9, 21, 70), and activates myeloid-derived suppressor cell (MDSCs) (Reference 71) and natural killer cell (NKs) (Reference 72) suppressor functions in the tumor microenvironment, suggesting that TNFR2 signaling has cell-specific and contextual-dependent immunological functions. Whether TNFR2 signaling in Tregs, MDSC, or NK cells influences host defense during S. aureus skin infections will be examined in future studies. Another finding was that TNFR2, but not TNFR1 mediated NOX2 activation and ROS production in the in vivo neutrophils. Both TNFR1 and TNFR2, however, contributed to ROS production in the in vitro neutrophils when co-cultured with S. aureus. This was unanticipated since TNFR1 harbors a cytosolic domain, which is not shared with TNFR2, that allows direct interactions with RFK to activate the NOX2 complex (Reference 73). Furthermore, in a preclinical model of colorectal tumors, TNFR1 signaling enhanced oxidative stress and tumor cell death by a mechanism that involved NOX-mediated ROS production (Reference 74). A possible explanation for our findings is that the cellular microenvironment of the neutrophil-rich abscess favors transmembrane TNF (mTNF) signaling rather than soluble TNF (sTNF) on neutrophils, as TNFR2 activation is dependent on receptor aggregation and has preferential sensitivity to mTNF present on cell surfaces (References 75, 76). We found that TNFR2, but not TNFR1, was responsible for in vivo NET formation during the S. aureus skin infection.

We discovered that TNFR2, but not TNFR1, was responsible for in vivo and in vitro NET formation in response to S. aureus exposure. Our observations align with previous in vitro studies that showed TNFR2-mediated pro-NETotic activity in human neutrophils (Reference 77) and mTNF-induced caspase- and death domain-independent cell death via mitochondrial ROS production (Reference 78). Another interesting finding was that TNFR2 controlled PAD4-dependent suicidal and vital NET formation during the S. aureus skin infection. Since suicidal NETs are ROS-dependent (References 79-81), our observation that TNFR2 regulates NOX2 activation and ROS production provides an explanation for the dependence of suicidal NET formation on TNFR2 signaling. In contrast, vital NETosis is independent of ROS production by NADPH oxidase (References 31, 38). Our finding that TNFR2 regulates vital NETosis aligns with a previous study that showed NADPH oxidase activity is dispensable in TNF-stimulated NET formation in vitro (Reference 82). Understanding the in vivo mechanisms of TNFR2-mediated suicidal and vital NET formation during S. aureus skin infections will be the focus of future work.

Interestingly, we found that TNFR2, but not PAD4 was critical for preventing systemic dissemination of the S. aureus skin infection, suggesting that NETs were dispensable for the containment of S. aureus in the skin. These results were unexpected since Yipp. et. al. found that NETs were essential for limiting S. aureus dissemination from the skin (Reference 38). This discrepancy may be explained by the differences in the skin infection models used (e.g., intradermal versus subcutaneous) and/or the methods to inhibit in vivo NET formation (e.g., PAD4 deficient mice versus DNase treatment). Regardless, our findings indicated that TNFR2 is crucial in controlling S. aureus spread from the skin in a mechanism independent of NET formation.

Lastly, we discovered that TNFR2 agonism acted as a novel immunotherapeutic strategy against bacterial skin infections, providing an important translational component to our findings. This anti-microbial efficacy was unanticipated since previous studies have used TNFR2 agonists to dampen pathogenic inflammatory responses in the context of neurodegeneration (References 86, 87), cancer (Reference 88), and acute graft-versus-host disease (References 48, 89), including the TNFR2 agonist used in this study (Reference 48). Importantly, we found that the TNFR2 agonist enhanced NET formation, in particular vital NETosis, which was associated with improved S. aureus clearance. Since TNFR2 agonist-treated mice did not have increased ROS in neutrophils, it is possible that the TNFR2 agonist acts by priming NADPH oxidase in neutrophils prior to S. aureus exposure in the skin. Thus, primed neutrophils can produce ROS more quickly in response to infection compared to vehicle treated neutrophils. Furthermore, TNFR2 agonism showed activity against Gram-positive and Gram-negative skin pathogens, indicating that this strategy has potential as a broad-spectrum anti-bacterial therapy. We also predict that TNFR1 agonism may act as a therapeutic target alone or additively in combination with the TNFR2 agonist, since our data suggests that TNFR1 and TNFR2 have non-redundant roles in inducing neutrophil recruitment and function, respectively. Additionally, our findings have important clinical implications beyond bacterial infections, specifically in the pathogenesis of inflammatory diseases exacerbated by NET formation (References 90, 91) and provide a potential mechanistic explanation for the efficacy of TNF inhibitors to dampen inflammation in these conditions.

Taken together, neutrophil-intrinsic TNFR1 and TNFR2 signaling orchestrated host defense against S. aureus skin infections in a mechanism that included neutrophil recruitment, NOX2-mediated ROS production, and NET formation. Furthermore, TNFR2 agonist treatment improved clearance of S. aureus and P. aeruginosa skin infections, which involved increased NET formation at the infection site. These findings provide important mechanistic insights and feasibility for agonistic targeting of TNFR2 as a novel immunotherapeutic strategy to treat antibiotic-resistant bacterial infections.

### 1.4 Methods

### 1.4.1 Study Design

The study's objective was to elucidate TNF-, TNFR1-, and TNFR2-mediated host defense mechanisms and to determine the efficacy and mechanism of action of a TNFR2 agonist as a host-direct immunotherapeutic for S. aureus skin infections. TNF-, TNFR1-, and TNFR2-mediated host defense mechanisms and the efficacy of TNFR2 agonism was investigated using an intradermal S. aureus skin infection mouse model whereby bacterial burden and skin lesions were longitudinally measured in vivo for 14 days. To understand TNF-, TNFR1-, and TNFR2-mediated host defense mechanisms, mice deficient in specific immune components were infected intradermally with S. aureus. To test the efficacy of TNFR2 agonist, WT mice were infected intradermally with S. aureus and treated with vehicle or TNFR2 agonist. The maximum neutrophil influx and TNF expression in WT mice were observed 3 days post-infection with S. aureus. Therefore, infected skin from 3 days post-infection was used to compare differential biological effects between WT, TNF^{-/-}, TNFR1^{-/-}, TNFR2^{-/-}, PAD4^{-/-}, vehicle-treated and TNFR2 agonist-treated mice. Cells isolated from infected mouse skin were profiled by multiparametric flow cytometry to identify changes in cell populations, cytokine and receptor expression, ROS production, and NET formation. Harvested skin biopsies from infected mice were also analyzed by histology for neutrophilic abscess formation and bacterial band length, and immunohistochemistry to quantify NET formation and NOX2 activation. Furthermore, without wishing to be bound to any one particular theory, it was thought that the TNFR2 agonist has broad-spectrum efficacy and evaluated the efficacy of the TNFR2 agonist against Gram-negative (P. aeruginosa) bacterial skin infections. The group sizes for each mouse strain included at least four mice per group with at least two iterations to confirm results, which was sufficient to ensure statistical significance, as previously described (References 16, 17). Each experimental group was paired with a respective control group to control for variation in bacterial inoculum between independent experiments. Outliers were identified and excluded by Grubb's test with Prism software (GraphPad 9 Software, La Jolla, California). Animal caretakers and researchers were not blinded to the study groups. All animal studies were approved by the Johns Hopkins University Animal Care and Use Committee.

### 1.4.2 Bacterial Strains

The bioluminescent S. aureus USA300 LAC::lux was derived from the parent S. aureus strain USA300 LAC, a well-described community-acquired methicillin-resistant S. aureus (CA-MRSA) clinical isolate that was obtained from an S. aureus skin and soft tissue infection outbreak in the Los Angeles County (LAC) Jail (References 16, 17, 94). The bioluminescent P. aeruginosa strain Xen41 (PerkinElmer) was obtained from the parental strain PAO1 (Reference 96). USA300 LAC::lux and Xen41 possess a modified luxABCDE operon from the bacterial insect pathogen, Photorhabdus luminescens, that the emission of blue-green light from live metabolically active bacteria is maintained in all progeny.

### 1.4.3 Bacterial Preparation

Bacterial strains were prepared as previously described (References 17, 18). S. aureus USA300 LAC::lux bacteria were streaked onto a tryptic soy agar (TSA) plate (tryptic soy broth [TSB] plus 1.5% bacto agar) and grown overnight at 37° C. in a bacterial incubator. Single colonies were picked and cultured in TSB at 37° C. in a shaking incubator (240 rpm) overnight (18 hours), followed by a 1:50 subculture at 37° C. for 2 hours to obtain mid-logarithmic phase bacteria. P. aeruginosa Xen41 bacteria were streaked onto a Luria-Bertani (LB) plate (LB broth plus 1.5% bacto agar) and grown overnight at 37° C. in a bacterial incubator. Single colonies of P. aeruginosa strain were grown overnight in LB broth at 37° C. shaking at 240 rpm, then diluted 1:50 and grown for 2.5 hours to obtain midlogarithmic growth phase bacteria. The bacteria were pelleted, washed 3 times, and resuspended in sterile phosphate-buffered saline (PBS) at the indicated concentrations for each bacterium below. The absorbance (A600) was measured to estimate the number of CFU, which was verified after overnight culture on TSA (S. aureus) and LB (P. aeruginosa) plates, respectively.

### 1.4.4 Bacterial Growth Curve Kinetics

Bacterial broth cultures of USA300 LAC::lux or Xen41 were prepared as described above. Overnight cultures were diluted 1:50 in their respective growth media. Next, 50 μL of bacterial cultures were incubated with either 50 μL vehicle (sterile PBS) or 50 μL of TNFR2 agonist to reach final concentrations of 80 μg/mL, 160 μg/mL, and 320 μg/mL in a total volume of 100 μL. The bacterial growth (OD600) was measured at 20 minutes intervals in triplicate for 10 hours while shaking at 282 cpm (counts per minute) at 37° C. in a Gen5 plate reader (BioTek).

### 1.4.5 Mice

6- to 10-week-old female wild-type (WT), TNF^{-/-}, TNFR1^{-/-}, TNFR2^{-/-}, and PAD4^{-/-} mice on a C57BL/6 background were obtained from Jackson Laboratories (Bar Harbor, ME) and used in all experiments for the mouse models of S. aureus intradermal infection. All mouse strains were bred and maintained under the same specific pathogen-free conditions, with air-isolated cages at an American Association for the Accreditation of Laboratory Animal Care (AAALAC)-accredited animal facility at Johns Hopkins University and handled according to procedures described in the Guide for the Care and Use of Laboratory Animals as well as Johns Hopkins University's policies and procedures as outlined in the Johns Hopkins University Animal Care and Use Training Manual.

### 1.4.6 Mouse Model of Intradermal Bacterial Skin Infection

A mouse model of S. aureus intradermal (i.d.) infection was performed as previously described (References 16-18). Briefly, the dorsal skin of anesthetized mice (2% isoflurane) was shaved and a 100 μL volume of PBS containing 3×10⁷ CFU of USA300 LAC::lux or 1×10⁶ CFU of Xen41 was intradermally injected. Total lesion size (cm²) was measured from digital photographs of the infected skin of anesthetized mice (2% isoflurane) by using the image analysis software program ImageJ (imagej.nih.gov/ij/) with a millimeter ruler as a reference.

### 1.4.7 In Vivo Bioluminescent Imaging (BLI)

In vivo bioluminescent imaging (BLI) was performed to approximate the in vivo bacterial burden, as previously described (References 17, 18). Briefly, anesthetized mice (2% isoflurane) were imaged using a Lumina III IVIS (PerkinElmer) and total flux (photons/s) was measured within a 1×10³ pixel square region of interest using Living Image software (PerkinElmer; limit of detection: 2×10⁴ photons/s) with USA300 LAC::lux or Xen41 bioluminescent bacterial strains. The bioluminescent signals detected from infected skin using USA300 LAC::lux closely approximated the actual bacterial burden measured by ex vivo CFU counting, as previously described (R2=0.9996)(Reference 19).

### 1.4.8 Mouse Serum and Skin Protein Isolation for EISA

Mouse blood was collected on the indicated days through retro-orbital bleeding using heparinized microhematocrit capillary tubes (Fisher Scientific) of anesthetized mice (2% isoflurane). Sera were collected by centrifuging the clotted blood for 10 minutes at 1500×g and 4° C. Sera were then frozen at −80° C. until needed. Mouse skin protein was collected by homogenizing a 10 mm skin punch biopsy in RIPA Lysis and Extraction Buffer (ThermoFisher Scientific) supplemented with Halt™ Protease and Phosphatase Inhibitor Cocktail (ThermoFisher Scientific). Skin protein was isolated by collecting the supernatant after centrifuging the skin homogenate (1500×g) for 5 minutes at 4° C. and frozen at −80° C. until needed. Mouse serum and skin TNF levels were quantified using a mouse TNF-alpha Quantikine ELISA Kit, according to the manufacturer's instructions (R&D Systems).

### 1.4.9 Er Vivo Kidney CFU Enumeration

Kidney bacterial CFU was obtained by isolating both kidneys of intradermally infected mice and homogenized with Pro200 Series homogenizer (Pro Scientific) in sterile PBS. Ex vivo CFUs of USA300 LAC::lux were counted after plating serially diluted kidney homogenates overnight on TSA plates.

### 1.4.10 Histologic Analysis of Mouse Skin

10-mm punch biopsy specimens were placed in 10% formalin and paraffin-embedded (FFPE). Skin cross-sections (4 μm) were prepared onto Superfrost Plus microscope slides (CardinalHealth) and stained with hematoxylin and eosin (H&E) or Gram stain by the Johns Hopkins Reference Histology Laboratory, according to clinical specimen guidelines. Skin sections were digitally scanned using a NanoZoomer XR (Hamamatsu, Japan) and images were exported at ×12.5 magnification with a millimeter-scale bar as a reference. Neutrophilic abscess area and bacterial band width were measured by brightfield microscopy images taken at ×12.5 magnification (Leica, DFC495) with a millimeter-scale bar as a reference using ImageJ software (NIH).

### 1.4.11 Flow Cytometry

10-millimeter punch biopsies of mouse dorsal skin were excised, minced, and placed in 3 mL RPMI containing 100 μg/mL DNase I (DN25, Sigma-Aldrich, St. Louis, MO) and 1.67 Wunsch units/mL Liberase TL (Roche, Basel, Switzerland). Single-cell suspensions were acquired by digesting the skin for 1 hour at 37° C. shaken at 70 rpm and filtering the digested samples through a 40-μm filter using a 3 mL syringe plunger. The cells were then washed once with RPMI and resuspended with PBS. The single-cell suspension was incubated with Viobility Fixable Dye (Miltenyi) to assess cell viability and TruStain FcX (BioLegend) to block FC receptor binding. The following antibodies were resuspended in PBS supplemented with 1% BSA and 5 mM EDTA (FACS buffer) to detect cell surface markers: VioBlue and APCVio770-anti-CD45 (REA737); PerCPVio700 and APC-anti-CD11b (REA592); PE, FITC, and APCVio770-anti-Ly6G (REA526); VioBlue and APCVio770-anti-Ly6C (REA796); PEVio770-anti-F4/80 (REA126); PE-anti-CD120a (TNFR1) (55R-286; BioLegend); PEVio770 and PerCPVio770-anti-CD120b (TNFR2) (REA252); PEVio770-anti-CD3 (REA641); PerCPVio700-anti-TCR7/6 (REA633); VioBlue-anti-CD4 (REA604); APC-anti-CD63 (REA563); PEVio770-anti-CD21/CD35 (REA800) (all from Miltenyi Biotec, otherwise indicated). Intracellular staining was performed by fixing and permeabilizing single-cell suspensions in BD Cytofix/Cytoperm for 20 minutes at 4° C. and then stained with FITC-anti-TNF (REA636, Miltenyi Biotec) and APC-anti-pro-IL-1β (REA577, Miltenyi Biotec). Intracellular staining for T cells was performed after 3 hours of stimulation with eBioscience™ Cell Stimulation Cocktail with protein transport inhibitors (ThermoFisher) at 37° C. Stimulated cells were fixed and permeabilized as previously described, and stained with PE-anti-IL17A (REA660, Miltenyi Biotec). Cell acquisition was performed on a MACSQuant flow cytometer (Miltenyi Biotec), and data were analyzed using MACSQuantify software (Miltenyi Biotec) and Cytobank software (Cytobank). Cell types were defined by flow cytometry according to the following gating strategies: monocytes (Ly6C+Ly6G−), macrophages (F4/80+), and neutrophils (Ly6C^{int/low}Ly6G+) were identified from the CD45+CD11b+ population from live single cells (FIG. 11). Non-hematopoietic cells were identified as the CD45− population from live single cells. TNF-, TNFR1- and TNFR2-expressing cells were first gated on live, single cells followed by surface markers to identify monocytes, macrophages, neutrophils, and non-hematopoietic cells as detailed above (FIG. 13).

For identifying suicidal and vital NETs, single cell suspension was incubated with Viobility Fixable Dye (Miltenyi) to assess cell viability and 5 μM of Sytox Orange (Invitrogen) to detect extracellular double-stranded DNA. Then, cells were fixed with BD Cytofix Fixation buffer for 20 minutes at 4° C. and stained with the following primary antibodies in FACS buffer. Anti-Histone H3 (citrulline R2+R8+R17) (H3-Cit) antibody (rabbit polyclonal, Abcam); Biotin Anti-Myeloperoxidase antibody (2D4, Abcam), VioBlue-anti-CD45 (REA737); PerCPVio700-anti-CD11b (REA592); APCVio770-anti-Ly6G (REA526); APC-anti-CD63 (REA563); PEVio770-anti-CD21/CD35 (REA800) (all from Miltenyi Biotec, otherwise indicated). Cells were washed and stained with secondary antibodies Donkey anti-Rabbit Alexa Fluor 647 (polyclonal, Abcam) and Alexa Fluor 488 Streptavidin (Biolegend) to detect surface H3-Cit and MPO, respectively. Suicidal and vital NETs were defined by flow cytometry according to the following gating strategies: neutrophils (CD11b+Ly6G+) were first gated from singlet CD45+CD11b+ cells. Then, suicidal and vital NETs were differentiated by dead and live neutrophils, respectively, followed by identification as MPO+H3-Cit+ neutrophils that had extracellular dsDNA (Sytox Orange+) (FIG. 14).

### 1.4.12 Immunofluorescence Microscopy

To identify NETs in the skin ex vivo, FFPE from day 3 post-infection were deparaffinized with heat-mediated antigen-retrieval in Trilogy buffer (Cell Marque) and blocked for 1 hour at room temperature with PBS supplemented with 0.5% Tween-20 (PBS-T) and 10% fetal bovine serum (FBS). Blocked slides were then incubated overnight at 4° C. with Rat-anti-Ly6G (2.5 μg/mL; 1A8, BD) and Rabbit-anti-H3-cit (2 μg/mL; polyclonal, Abcam) diluted in 10% FBS PBS-T buffer. Stained slides were washed three times with PBS-T and incubated first for 1 hour at room temperature with secondary antibodies goat anti-rat IgG Alexa Fluor 594 (2 μg/mL; Invitrogen) and goat anti-rabbit IgG Alexa Fluor 488 (2 μg/mL; Invitrogen) diluted in 10% FBS PBS-T buffer. After secondary antibody incubation, slides were washed three times with PBS-T, as previously described. Nuclei were stained with DAPI (1 μg/mL in PBS) for 5 min, washed twice with PBS, and subsequently mounted in ProLong Diamond Antifade Mountant (ThermoFisher Scientific).

To determine activated p47ₚₕₒₓ in the skin ex vivo, FFPE skin sections were deparaffinized and blocked for 1 hour at room temperature with PBS-T supplemented with 1% bovine serum albumin (BSA). Skin sections were then stained with Rat-anti-Ly6G (2.5 μg/mL; 1A8, BD) and Rabbit-anti-phosphorylated p47ₚₕₒₓ (pSer345) (10 μg/mL; polyclonal, Sigma) diluted in 0.5% BSA PBS-T overnight at 4° C. Stained slides were washed three times with PBS-T and incubated first for 1 hour at room temperature with secondary antibodies goat anti-rat IgG Alexa Fluor 594 (2 μg/mL; Invitrogen) and goat anti-rabbit IgG Alexa Fluor 488 (2 μg/mL; Invitrogen) diluted in 0.5% BSA PBS-T buffer. Nuclei were stained with DAPI (1 μg/mL in PBS) for 5 min, washed twice with PBS, and subsequently mounted in ProLong Diamond Antifade Mountant (ThermoFisher Scientific).

### 1.4.13 Immunofluorescence Quantification

For H3-Cit quantification, at least three images of the neutrophilic abscess (defined by positive Ly6G stain) for each skin section were taken at ×400 magnification using a fluorescence microscope (DFC365FX, Leica Microsystems). The fluorescence intensity of H3-cit stain was quantified by averaging the integrated density of whole images using ImageJ software (NIH).

For activated p47ₚₕₒₓ quantification, at least 8 images of the neutrophilic abscess for each skin section were taken at ×400 magnification using a fluorescence microscope (DFC365FX, Leica Microsystems). For each image, the number of punctate stains was quantified by normalizing the total count of punctate stains to the area of whole image using ImageJ software (NIH). Then, the total number of activated p47ₚₕₒₓ per skin section was determined by averaging the number of punctate stains from all images acquired for each skin section.

### 1.4.14 Bone Marrow Reconstitution Generation

WT or TNF^{-/-} mice were given a lethal dose of 8Gy irradiation with the CIXD Biological Irradiator (XStrahl), after which 1×10⁷ bone marrow (BM) cells were administered via retroorbital vein injection within 3 hours following irradiation. For 3 weeks after irradiation, mice were given drinking water supplemented with Elanco Baytril 100 (0.5 mg/mL; Elanco Animal Health). After 7 to 8 weeks following irradiation, BM reconstitution was confirmed by flow cytometric analysis for TNF-producing cells in the blood.

### 1.4.15 Neutrophil Isolation and Adoptive Transfer to Mice

To isolate neutrophils, a single-cell suspension of bone marrow (BM) cells was obtained from the femurs and tibias of 8- to 10-week old WT, TNF^{-/-}, TNFR1^{-/-}-, or TNFR2^{-/-} mice by flushing with ≥5 mL of sterile 4° C. RPMI media using a 25 G needle syringe. BM cells were strained over a 40 μm filter, counted, and resuspended in MACS Separation Buffer (degassed PBS containing BSA [0.5%] and EDTA [2 mM]) (Miltenyi Biotec) to a final concentration of 2.5×10⁸ cells/mL. Neutrophils were then purified using a Neutrophil Isolation Kit for mice, according to the manufacturer's instructions (Miltenyi Biotec). After purification, the cells were >94% viable and >92% CD45+CD11b+Ly6G+Ly6C− neutrophils (FIG. 11). For adoptive transfer experiments, 5.0×10⁶ purified neutrophils resuspended in sterile 4° C. PBS were injected intravenously via the retroorbital vein 2 hours prior to S. aureus skin infection.

### 1.4.16 In Vitro Neutrophil ROS Quantification

Neutrophils were isolated from BM cells as previously described (refer to Neutrophil isolation and adoptive transfer to mice). Isolated neutrophils were resuspended to a final concentration of 2.0×10⁶ cells/mL in RPMI supplemented with 10% FBS for ROS detection. Cells were seeded into 96-well U-bottom plates at 2.0×10⁵ cells in 100 μL per well.

S. aureus USA300 LAC::lux bacteria was prepared as previously described (refer to Bacterial preparation). Multiplicity of infection (MOI) of 100 (2.0×10⁷ CFU/mL) were prepared in RPMI supplemented with 10% FBS. Triplicate wells of neutrophils were cultured for 2 hours at 37° C. with the following reagents: RPMI supplemented with 10% FBS, Phorbol myristate acetate (PMA) at 100 ng/mL. S. aureus MOI 100. After stimulation, cells were stained with 100 μL of 20 μM of DCFDA dye (Abcam) in RPMI supplemented with 10% FBS for 30 minutes at 37° C. Subsequently, cells were incubated with Viobility Fixable Dye (Miltenyi) to assess cell viability. After staining, cells were washed once and resuspended in 200 μL of FACS buffer for acquisition on the flow cytometer.

### 1.4.17 In Vitro Neutrophil NET Quantification

For NET detection, neutrophils were resuspended to a final concentration of 2.0×10⁶ cells/mL in R1 medium (RPMI supplemented with 1×MEM, 1% penicillin/streptomycin, and 1% FBS). Cells were seeded into black 96-well flat-bottom plates at 2.0×10⁵ cells in 100 μL per well.

S. aureus USA300 LAC::lux bacteria was prepared as previously described (refer to Bacterial preparation). Multiplicity of infection (MOI) of 100 (2.0×10⁷ CFU/mL) were prepared in R1 medium. Triplicate wells of neutrophils were cultured for 2 hours with the following reagents: R1 medium, 100 ng/mL Phorbol myristate acetate (PMA), S. aureus MOI 100. After 2 hours of stimulation, cells were stained with 50 μL of 5 μM SYTOX Green (ThermoFisher) in R1 medium for 5 minutes at room temperature. Cells were then incubated with Viobility Fixable Dye (Miltenyi) to assess cell viability. After staining, cells were washed once and resuspended in 200 μL of FACS buffer for acquisition on the flow cytometer.

### 1.4.18 TNFR2 Agonist and Administration

NewSTAR2 (TNFR2 agonist) was generated as previously described (Reference 48). Briefly, the TNFR2 agonist NewSTAR2 comprises two single-chain encoded TNFR2-specific murine TNF mutant trimers (sc(mu)TNF80) fused to the C-termini of an IgG1 molecule of irrelevant specificity in mice and the N297A mutation that minimizes interaction with Fcγ-receptors. The purified TNFR2 agonist was LPS-free and in the working concentration of 1.4-1.6 mg/mL for in vivo experiments. After 4 hours post-infection, mice were injected intraperitoneally (i.p.) with 100-115 μL of a single dose of 8 mg/kg TNFR2 agonist or vehicle (sterile PBS).

### 1.4.19 Statistical Analysis

Data comparison between two groups for longitudinal comparisons was compared using a 2-way analysis of variance (ANOVA). Single comparisons were compared by using a nonparametric 2-tailed Student's t test or a nonparametric Mann-Whitney test. Data between multiple groups (≥3 groups) were compared using a 1-way ANOVA multiple comparisons test with Tukey correction or Kruskal-Wallis test with Dunn's correction as indicated in figure legends. All statistical analysis was calculated with Prism software (GraphPad 9 Software, La Jolla, California). Data are presented as means A standard error of the mean (SEM). P<0.05 was considered statistically significant.

### 1.4.20 Study Approval

All animal experiments were approved by the Johns Hopkins University Animal Care and Use Committee.

### 1.4.21 RNA Isolation and Quantitative Real-Time PCR (qRT-PCR)

Skin punch biopsy (10-millimeter) were snap-frozen in liquid nitrogen and homogenized (Pro200 Series homogenizer, Pro Scientific) in TRIzol reagent. 10% of TRIzol volume of chloroform was vortexed with homogenized skin until cloudy and incubated on ice for 5 minutes. The samples were then centrifuged at 12000×g for 15 minutes at 4° C. to phase separate the phenol-chloroform phase, interphase, and upper aqueous phase. RNA isolation was performed on the upper aqueous layer using the Direct-zol RNA-Mini Prep kit according to the manufacturer's instructions (Zymo Research). The final RNA elution volume was determined by the mass of the tissue (1 μL of elution buffer per 1 mg of tissue). cDNA was synthesized using a High Capacity RNA-to-cDNA Kit according to the manufacturer's instructions (Applied Biosystems). Commercially available primers and probes for mouse Cxcl1, Cxcl2, Cxcl3, Cxcl5, Mpo, S100a8, S100a9, Il1b, Il6, Tnf, and the Taqman Gene Expression Master Mix were used (Applied Biosystems). Quantitative real-time PCR (qRT-PCR) was performed using a CFX96 Real-time PCR system (Bio-Rad). The relative quantities of mRNA per sample were determined using the ΔΔC(T) formula and normalized to WT Cq value (A.U.).

### 1.4.22 In Vivo Antibody Administration

To block TNFR2, mice were given intraperitoneal injection (i.p.) with anti-TNFR2 antibody (clone TR75-54.7) 500 μg in 100 p sterile PBS beginning on day −1, day 0, and day 1 of S. aureus skin infection and then every other day throughout the 14-days of observation, according to dose recommendation from references (References 105, 106) on the data sheet provided by the manufacturer (BioXCell). The mice in the control group were treated i.p. with InVivoMAb polyclonal Armenian hamster IgG isotype control antibody (BioXCell) at the equivalent dose and volume.

### 1.4.23 RNA Isolation and RNA Sequencing

Skin punch biopsy (10-millimeter) was immediately placed in RNAlater (Sigma), snap-frozen in liquid nitrogen, and homogenized (Pro200 Series homogenizer, Pro Scientific) in TRIzol reagent. 10% of TRIzol volume of chloroform was vortexed with homogenized skin until cloudy and incubated on ice for 5 minutes. The samples were then centrifuged at 12000×g for 15 minutes at 4° C. to phase separate the phenol-chloroform phase, interphase, and upper aqueous phase. RNA isolation was performed on the upper aqueous layer using the Direct-zol RNA-Mini Prep kit according to the manufacturer's instructions (Zymo Research). The final RNA elution volume was determined by the mass of the tissue (1 μL of elution buffer per 1 mg of tissue). Frozen RNA was shipped to BGI for RNA quality control and RNA sequencing. Reads were aligned with the HISAT algorithm to the Mus musculus GCF_000001635.26_GRCm38.p6 reference genome from the NCBI database. Differentially expressed genes (DEG) were determined between WT and TNFR2^{-/-} groups with DESeq2 using default parameters (References 107, 108). Counts were quantified and normalized to fragments per kilobase per million mapped reads (FPKM) using BGI Dr. Tom, and a nonparametric 2-tailed Student's t test was run between WT and TNFR2^{-/-} groups. Data were visualized using GraphPad Prism 9.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.
1. Turner N A, Sharma-Kuinkel B K, Maskarinec S A, Eichenberger E M, Shah P P, Carugati M, et al. Methicillin-resistant Staphylococcus aureus: an overview of basic and clinical research. Nat Rev Microbiol. 2019; 17(4):203-18.
2. Collaborators A R. Global burden of bacterial antimicrobial resistance in 2019: a systematic analysis. Lancer. 2022; 399(10325):629-55.
3. Tong S Y, Davis J S, Eichenberger E, Holland T L, and Fowler V G. Staphylococcus aureus infections: epidemiology, pathophysiology, clinical manifestations, and management. Clin Microbiol Rev. 2015; 28(3):603-61.
4. Kaye K S, Petty L A, Shorr A F, and Zilberberg M D. Current Epidemiology, Etiology, and Burden of Acute Skin Infections in the United States. Clin Infect Dis. 2019; 68(Suppl 3):S193-S9.
5. Gough P, and Myles I A. Tumor Necrosis Factor Receptors: Pleiotropic Signaling Complexes and Their Differential Effects. Front Immunol. 2020; 11:585880.
6. Brenner D, Blaser H, and Mak T W. Regulation of tumour necrosis factor signalling: live or let die. Nat Rev Immunol. 2015; 15(6):362-74.
7. Tracey D, Klareskog L, Sasso E H, Salfeld J G, and Tak P P. Tumor necrosis factor antagonist mechanisms of action: a comprehensive review. Pharmacol Ther. 2008; 117(2):244-79.
8. Wajant H, and Siegmund D. TNFR1 and TNFR2 in the Control of the Life and Death Balance of Macrophages. Front Cell Der Biol. 2019; 7:91.
9. Yang S, Xie C, Chen Y, Wang J, Chen X, Lu Z, et al. Differential roles of TNFα-TNFR1 and TNFα-TNFR2 in the differentiation and function of CD4. Cell Death Dis. 2019; 10(1):27.
10. Ticha O, Slanina P, Moos L, Stichova J, Vlkova M, and Bekeredjian-Ding I. TNFR2 expression is a hallmark of human memory B cells with suppressive function. Eur J Immunol. 2021; 51(5):1195-205.
11. Wang F, Schwarz B T, Graham W V, Wang Y, Su L, Clayburgh D R, et al. IFN-gamma-induced TNFR2 expression is required for TNF-dependent intestinal epithelial barrier dysfunction. Gastroenterology. 2006; 131(4):1153-63.
12. Suto T, Tosevska A, Dalwigk K, Kugler M, Dellinger M, Stanic I, et al. TNFR2 is critical for TNF-induced rheumatoid arthritis fibroblast-like synoviocyte inflammation. Rheumatology (Oxford). 2022.
13. Gerriets V G A, Khaddour K. StatPearls Publishing, Treasure Island (FL); 2022.
14. Frdling E, Baumann C, Cuny J F, Bigard M A, Schmutz J L, Barbaud A, et al. Cumulative incidence of, risk factors for, and outcome of dermatological complications of anti-TNF therapy in inflammatory bowel disease: a 14-year experience. Am J Gastroenterol. 2015; 110(8):1186-96.
15. Cossio M L, Genois A, Jantchou P, Hatami A, Deslandres C, and McCuaig C. Skin Manifestations in Pediatric Patients Treated With a TNF-Alpha Inhibitor for Inflammatory Bowel Disease: A Retrospective Study [Formula: see text]. J Cutan Med Surg. 2020; 24(4):333-9.
16. Marchitto M C, Dillen C A, Liu H, Miller R J, Archer N K, Ortines R V, et al. Clonal Vγ6+Vδ4+ T cells promote IL-17-mediated immunity against Staphylococcus aureus skin infection. Proc Nat Acad Sci USA. 2019; 116(22):10917-26.
17. Dillen C A, Pinsker B L, Marusina A I, Merleev A A, Farber O N, Liu H, et al. Clonally expanded γδ T cells protect against Staphylococcus aureus skin reinfection. J Clin Invest. 2018; 128(3):1026-42.
18. Alphonse M P, Rubens JH, Ortines R V, Orlando N A, Patel A M, Dikeman D, et al. Pan-caspase inhibition as a potential host-directed immunotherapy against MRSA and other bacterial skin infections. Sci Transl Med. 2021; 13(601).
19. Guo Y, Ramos R I, Cho J S, Donegan N P, Cheung A L, and Miller L S. In vivo bioluminescence imaging to evaluate systemic and topical antibiotics against community-acquired methicillin-resistant Staphylococcus aureus-infected skin wounds in mice. Antimicrob Agents Chemother. 2013; 57(2):855-63.
20. Sedger L M, and McDermott M F. TNF and TNF-receptors: From mediators of cell death and inflammation to therapeutic giants—past, present and future. Cytokine Growth Factor Rev. 2014; 25(4):453-72.
21. Mehta A K, Gracias D T, and Croft M. TNF activity and T cells. Cytokine. 2018; 101:14-8.
22. Dudeck J, Kotrba J, Immler R, Hoffmann A, Voss M, Alexaki V I, et al. Directional mast cell degranulation of tumor necrosis factor into blood vessels primes neutrophil extravasation. Immunity. 2021; 54(3):468-83.e5.
23. Uhlén M, Fagerberg L, Hallström BM, Lindskog C, Oksvold P, Mardinoglu A, et al. Proteomics. Tissue-based map of the human proteome. Science. 2015; 347(6220):1260419.
24. Jiang Y, Tsoi LC, Billi A C, Ward N L, Harms P W, Zeng C, et al. Cytokinocytes: the diverse contribution of keratinocytes to immune responses in skin. JCI Insight. 2020; 5(20).
25. Cho J S, Pietras E M, Garcia N C, Ramos R I, Farzam D M, Monroe H R, et al. IL-17 is essential for host defense against cutaneous Staphylococcus aureus infection in mice. J Clin Invest. 2010; 120(5):1762-73.
26. Cho J S, Guo Y, Ramos R I, Hebroni F, Plaisier S B, Xuan C, et al. Neutrophil-derived IL-1β is sufficient for abscess formation in immunity against Staphylococcus aureus in mice. PLoS Pathog. 2012; 8(11):e1003047.
27. Miller L S, Pietras E M, Uricchio L H, Hirano K, Rao S, Lin H, et al. Inflammasome-mediated production of IL-1beta is required for neutrophil recruitment against Staphylococcus aureus in vivo. J Immunol. 2007; 179(10):6933-42.
28. Matsumoto M, Nakagawa S, Zhang L, Nakamura Y, Villaruz A E, Otto M, et al. Interaction between Staphylococcus Agr virulence and neutrophils regulates pathogen expansion in the skin. Cell Host Microbe. 2021; 29(6):930-40.e4.
29. Khandpur R, Carmona-Rivera C, Vivekanandan-Giri A, Gizinski A, Yalavarthi S, Knight J S, et al. NETs are a source of citrullinated autoantigens and stimulate inflammatory responses in rheumatoid arthritis. Sci Transl Med. 2013; 5(178):178ra40.
30. Palmer L J, Damgaard C, Holmstrup P, and Nielsen C H. Influence of complement on neutrophil extracellular trap release induced by bacteria. J Periodontal Res. 2016; 51(1):70-6.
31. Pilsczek F H, Salina D, Poon K K, Fahey C, Yipp B G, Sibley C D, et al. A novel mechanism of rapid nuclear neutrophil extracellular trap formation in response to Staphylococcus aureus. J Immunol. 2010; 185(12):7413-25.
32. Papayannopoulos V. Neutrophil extracellular traps in immunity and disease. Nat Rev Immunol. 2018; 18(2):134-47.
33. Rohrbach A S, Slade D J, Thompson P R, and Mowen K A. Activation of PAD4 in NET formation. Front Immunol. 2012; 3:360.
34. D′Cruz A A, Speir M, Bliss-Moreau M, Dietrich S, Wang S, Chen A A, et al. The pseudokinase MLKL activates PAD4-dependent NET formation in necroptotic neutrophils. Sci Signal. 2018; 11(546).
35. Neeli I, Khan S N, and Radic M. Histone deimination as a response to inflammatory stimuli in neutrophils. J Immunol. 2008; 180(3):1895-902.
36. Brinkmann V, Abu Abed U, Goosmann C, and Zychlinsky A. Immunodetection of NETs in Paraffin-Embedded Tissue. Front Immunol. 2016; 7:513.
37. Kaplan M J, and Radic M. Neutrophil extracellular traps: double-edged swords of innate immunity. J Immunol. 2012; 189(6):2689-95.
38. Yipp B G, Petri B, Salina D, Jenne C N, Scott B N, Zbytnuik L D, et al. 15 Infection-induced NETosis is a dynamic process involving neutrophil multitasking in vivo. Nat Med. 2012; 18(9):1386-93.
39. von Köckritz-Blickwede M, and Winstel V. Molecular Prerequisites for Neutrophil Extracellular Trap Formation and Evasion Mechanisms of Staphylococcus aureus. Front Immunol. 2022; 13:836278.
40. Van Avondt K, and Hartl D. Mechanisms and disease relevance of neutrophil extracellular trap formation. Eur J Clin Invest. 2018; 48 Suppl 2:e12919.
41. von Köckritz-Blickwede M, and Winstel V. Molecular Prerequisites for Neutrophil Extracellular Trap Formation and Evasion Mechanisms of. Front Immunol. 2022; 13:836278.
42. Monteith A J, Miller J M, Maxwell C N, Chazin W J, and Skaar E P. Neutrophil extracellular traps enhance macrophage killing of bacterial pathogens. Sci Adv. 2021; 7(37):eabj2101.
43. Castanheira F V S, and Kubes P. Neutrophils and NETs in modulating acute and chronic inflammation. Blood. 2019; 133(20):2178-85.
44. Dahlgren C, Karlsson A, and Bylund J. Intracellular Neutrophil Oxidants: From Laboratory Curiosity to Clinical Reality. J Immunol. 2019; 202(11):3127-34.
45. Parker H, and Winterbourn C C. Reactive oxidants and myeloperoxidase and their involvement in neutrophil extracellular traps. Front Immunol. 2012; 3:424.
46. Lambeth J D. NOX enzymes and the biology of reactive oxygen. Nat Rev Immunol. 2004; 4(3):181-9.
47. Rastogi R, Geng X, Li F, and Ding Y. NOX Activation by Subunit Interaction and Underlying Mechanisms in Disease. Front Cell Neurosci. 2016; 10:301.
48. Vargas J G, Wagner J, Shaikh H, Lang I, Medler J, Anany M, et al. A TNFR2-Specific TNF Fusion Protein With Improved In Vivo Activity. Front Immunol. 2022; 13:888274.
49. Ki V, and Rotstein C. Bacterial skin and soft tissue infections in adults: A review of their epidemiology, pathogenesis, diagnosis, treatment and site of care. Can J Infect Dis Med Microbiol. 2008; 19(2):173-84.
50. Miller R J, Crosby H A, Schilcher K, Wang Y, Ortines R V, Mazhar M, et al. Development of a Staphylococcus aureus reporter strain with click beetle red luciferase for enhanced in vivo imaging of experimental bacteremia and mixed infections. Sci Rep. 2019; 9(1):16663.
51. Kalliolias G D, and Ivashkiv L B. TNF biology, pathogenic mechanisms and emerging therapeutic strategies. Nat Rev Rheumatol. 2016; 12(1):49-62.
52. Varley C D, Deodhar A A, Ehst B D, Bakke A, Blauvelt A, Vega R, et al. Persistence of Staphylococcus aureus colonization among individuals with immune-mediated inflammatory diseases treated with TNF-α inhibitor therapy. Rheumatology (Oxford). 2014; 53(2):332-7.
53. Wang Y, Ashbaugh A G, Dikeman D A, Zhang J, Ackerman N E, Kim S E, et al. Interleukin-1β and tumor necrosis factor are essential in controlling an experimental orthopedic implant-associated infection. J Orthop Res. 2020; 38(8):1800-9.
54. Nakane A, Okamoto M, Asano M, Kohanawa M, and Minagawa T. Endogenous gamma interferon, tumor necrosis factor, and interleukin-6 in Staphylococcus aureus infection in mice. Infect Immun. 1995; 63(4):1165-72.
55. Kielian T, Bearden E D, Baldwin A C, and Esen N. IL-1 and TNF-alpha play a pivotal role in the host immune response in a mouse model of Staphylococcus aureus-induced experimental brain abscess. J Neuropathol Exp Neurol. 2004; 63(4):381-96.
56. Stenzel W, Soltek S, Miletic H, Hermann M M, Karner H, Sedgwick J D, et al. An essential role for tumor necrosis factor in the formation of experimental murine Staphylococcus aureus-induced brain abscess and clearance. J Neuropathol Exp Neurol. 2005; 64(1):27-36.
57. Gideon H P, Phuah J, Junecko B A, and Mattila J T. Neutrophils express pro- and anti-inflammatory cytokines in granulomas from Mycobacterium tuberculosis-infected cynomolgus macaques. Mucosal Immunol. 20109; 12(6):1370-81.
58. Grivennikov S I, Tumanov A V, Liepinsh D J, Kruglov A A, Marakusha B I, Shakhov A N, et al. Distinct and nonredundant in vivo functions of TNF produced by t cells and macrophages/neutrophils: protective and deleterious effects. Immunity. 2005; 22(1):93-104.
59. Arnott C H, Scott K A, Moore R J, Robinson S C, Thompson R G, and Balkwill F R. Expression of both TNF-alpha receptor subtypes is essential for optimal skin tumour development. Oncogene. 2004; 23(10):1902-10.
60. Miller L S, O'Connell R M, Gutierrez M A, Pietras E M, Shahangian A, Gross C E, et al. MyD88 mediates neutrophil recruitment initiated by IL-1R but not TLR2 activation in immunity against Staphylococcus aureus. Immunity. 2006; 24(1):79-91.
61. Tranchemontagne Z R, Camire R B, O'Donnell V J, Baugh J, and Burkholder K M. Staphylococcus aureus Strain USA300 Perturbs Acquisition of Lysosomal Enzymes and Requires Phagosomal Acidification for Survival inside Macrophages. Infect Immun. 2016; 84(1):241-53.
62. Land A D, Hogan P, Fritz S, and Levin P A. Phenotypic Variation Is Almost Entirely Independent of the Host-Pathogen Relationship in Clinical Isolates of S. aureus. PLoS One. 2015; 10(6):e0129670.
63. Strobel M, Pförtner H, Tuchscherr L, Völker U, Schmidt F, Kramko N, et al. Post-invasion events after infection with Staphylococcus aureus are strongly dependent on both the host cell type and the infecting S. aureus strain. Clin Microbiol Infect. 2016; 22(9):799-809.
64. Gómez MI, Lee A, Reddy B, Muir A, Soong G, Pitt A, et al. Staphylococcus aureus protein A induces airway epithelial inflammatory responses by activating TNFR1. Nat Med. 2004; 10(8):842-8.
65. Ledo C, Gonzalez C D, Garofalo A, Sabbione F, Keitelman I A, Giai C, et al. Protein A Modulates Neutrophil and Keratinocyte Signaling and Survival in Response to Staphylococcus aureus. Front Immunol. 2020; 11:524180.
66. Giai C, Gonzalez C, Ledo C, Garofalo A, Di Genaro M S, Sordelli D O, et al. Shedding of tumor necrosis factor receptor 1 induced by protein A decreases tumor necrosis factor alpha availability and inflammation during systemic Staphylococcus aureus infection. Infect Immun. 2013; 81(11):4200-7.
67. Gómez MI, O'Seaghdha M, Magargee M, Foster T J, and Prince A S. Staphylococcus aureus protein A activates TNFR1 signaling through conserved IgG binding domains. J Biol Chem. 2006; 281(29):20190-6.
68. Bai J, Ding B, and Li H. Targeting TNFR2 in Cancer: All Roads Lead to Rome. Front Immunol. 2022; 13:844931.
69. Salomon B L. Insights into the biology and therapeutic implications of TNF and regulatory T cells. Nat Rev Rheumatol. 2021; 17(8):487-504.
70. Cicchese J M. Evans S, Hult C, Joslyn L R, Wessler T, Millar J A, et al. Dynamic balance of pro- and anti-inflammatory signals controls disease and limits pathology. Immunol Rev. 2018; 285(1):147-67.
71. Hu X, Li B, Li X, Zhao X, Wan L, Lin G, et al. Transmembrane TNF-α promotes suppressive activities of myeloid-derived suppressor cells via TNFR2. J Immunol. 2014; 192(3):1320-31.
72. Ivagnés A, Messaoudene M, Stoll G, Routy B, Fluckiger A, Yamazaki T, et al. TNFR2/BIRC3-TRAF1 signaling pathway as a novel NK cell immune checkpoint in cancer. Oncoimmunology. 2018; 7(12):e1386826.
73. Blaser H, Dostert C, Mak T W, and Brenner D. TNF and ROS Crosstalk in Inflammation. Trends Cell Biol. 2016; 26(4):249-61.
74. Habtetsion T, Ding Z C, Pi W, Li T, Lu C, Chen T, et al. Alteration of Tumor Metabolism by CD4+ T Cells Leads to TNF-α-Dependent Intensification of Oxidative Stress and Tumor Cell Death. Cell Metab. 2018; 28(2):228-42.e6.
75. Grell M, Douni E, Wajant H, Löhden M, Clauss M, Maxeiner B, et al. The transmembrane form of tumor necrosis factor is the prime activating ligand of the 80 kDa tumor necrosis factor receptor. Cell. 1995; 83(5):793-802.
76. Ranzinger J, Krippner-Heidenreich A, Haraszti T, Bock E, Tepperink J, Spatz J P, et al. Nanoscale arrangement of apoptotic ligands reveals a demand for a minimal lateral distance for efficient death receptor activation. Nano Lett. 2009; 9(12):4240-5.
77. Neuenfeldt F, Schumacher J C, Grieshaber-Bouyer R, Habicht J, Schröder-Braunstein J, Gauss A, et al. Inflammation induces pro-NETotic neutrophils via TNFR2 signaling. Cell Rep. 2022; 39(3):110710.
78. Ardestani S, Deskins D L, and Young P P. Membrane TNF-alpha-activated programmed necrosis is mediated by Ceramide-induced reactive oxygen species. J Mol Signal. 2013; 8(1):12.
79. Fuchs T A, Abed U, Goosmann C, Hurwitz R, Schulze I, Wahn V, et al. Novel cell death program leads to neutrophil extracellular traps. J Cell Biol. 2007; 176(2):231-41.
80. Bianchi M, Hakkim A, Brinkmann V, Siler U, Seger R A, Zychlinsky A, et al. Restoration of NET formation by gene therapy in CGD controls aspergillosis. Blood. 2009; 114(13):2619-22.
81. Lim M B, Kuiper J W, Katchky A, Goldberg H, and Glogauer M. Rac2 is required for the formation of neutrophil extracellular traps. J Leukoc Biol. 2011; 90(4):771-6.
82. Tatsiy O, and McDonald P P. Physiological Stimuli Induce PAD4-Dependent, ROS-Independent NETosis, With Early and Late Events Controlled by Discrete Signaling Pathways. Front Immunol. 2018; 9:2036.
83. Rodríguez-Espinosa O, Rojas-Espinosa O, Moreno-Altamirano M M, López-Villegas E O, and Sánchez-García FJ. Metabolic requirements for neutrophil extracellular traps formation. Immunology. 2015; 145(2):213-24.
84. Li D D, Jawale C V, Zhou C, Lin L, Trevejo-Nunez G J, Rahman S A, et al. Fungal sensing enhances neutrophil metabolic fitness by regulating antifungal Glut1 activity. Cell Host Microbe. 2022; 30(4):530-44.e6.
85. de Kivit S, Mensink M, Hoekstra A T, Berlin I, Derks R J E, Both D, et al. Stable human regulatory T cells switch to glycolysis following TNF receptor 2 costimulation. Nat Metab. 2020; 2(10):1046-61.
86. Fischer R, Maier O, Siegemund M, Wajant H, Scheurich P, and Pfizenmaier K. A TNF receptor 2 selective agonist rescues human neurons from oxidative stress-induced cell death. PLoS One. 2011; 6(11):e27621.
87. Ortí-Casañ N, Zuhorn I S, Naudé PJW, De Deyn P P, van Schaik P E M, Wajant H, et al. A TNF receptor 2 agonist ameliorates neuropathology and improves cognition in an Alzheimer's disease mouse model. Proc Natl Acad Sci USA. 2022; 119(37):e2201137119.
88. Medler J, Kucka K, and Wajant H. Tumor Necrosis Factor Receptor 2 (TNFR2): An Emerging Target in Cancer Therapy. Cancers (Basel). 2022; 14(11).
89. Chopra M, Biehl M, Steinfatt T, Brandl A, Kums J, Amich J, et al. Exogenous TNFR2 activation protects from acute GvHD via host T reg cell expansion. J Exp Med. 2016; 213(9):1881-900.
90. Demers M, and Wagner D D. NETosis: a new factor in tumor progression and cancer-associated thrombosis. Semin Thromb Hemost. 2014; 40(3):277-83.
91. Swystun L L, Mukherjee S, and Liaw P C. Breast cancer chemotherapy induces the release of cell-free DNA, a novel procoagulant stimulus. J Thromb Haemost. 2011; 9(11):2313-21.
92. Yang S, Wang J, Brand D D, and Zheng S G. Role of TNF-TNF Receptor 2 Signal in Regulatory T Cells and Its Therapeutic Implications. Front Immunol. 2018; 9:784.
93. Ruddle N H. Lymphotoxin and TNF: how it all began—a tribute to the travelers. Cytokine Growth Factor Rev. 2014; 25(2):83-9.
94. Thurlow L R, Hanke M L, Fritz T, Angle A, Aldrich A, Williams S H, et al. Staphylococcus aureus biofilms prevent macrophage phagocytosis and attenuate inflammation in vivo. J Immunol. 2011; 186(11):6585-96.
95. Davis R W, Eggleston H, Johnson F, Nahrendorf M, Bock P E, Peterson T, et al. In Vivo Tracking of Streptococcal Infections of Subcutaneous Origin in a Murine Model. Mol Imaging Biol. 2015; 17(6):793-801.
96. Thompson J M, Miller R J, Ashbaugh A G, Dillen C A, Pickett J E, Wang Y, et al. Mouse model of Gram-negative prosthetic joint infection reveals therapeutic targets. JCI Insight. 2018; 3(17).
97. C. Johansen, T. Bertelsen, C. Ljungberg, M. Mose, L. Iversen, Characterization of TNF-α- and IL-17A-Mediated Synergistic Induction of DEFB4 Gene Expression in Human Keratinocytes through IκBζ. J Invest Dermatol 136, 1608-1616 (2016).
98. C. Tecchio, A. Micheletti, M. A. Cassatella, Neutrophil-derived cytokines: facts beyond expression. Front Immunol 5, 508 (2014).
99. M. A. Cassatella, On the production of TNF-related apoptosis-inducing ligand (TRAIL/Apo-2L) by human neutrophils. J Leukoc Biol 79, 1140-1149 (2006).
100. N. C. Di Paolo et al., Interdependence between Interleukin-1 and Tumor Necrosis Factor Regulates TNF-Dependent Control of Mycobacterium tuberculosis Infection. Immunity 43, 1125-1136 (2015).
101. S. Iwamoto et al., TNF-alpha drives human CD14+ monocytes to differentiate into CD70+ dendritic cells evoking Th1 and Th17 responses. J Immunol 179, 1449-1457 (2007).
102. A. Othman, M. Sekheri, J. G. Filep, Roles of neutrophil granule proteins in orchestrating inflammation and immunity. FEBS J 289, 3932-3953 (2022).
103. K. R. McLeish et al., Frontline Science: Tumor necrosis factor-α stimulation and priming of human neutrophil granule exocytosis. J Leukoc Biol 102, 19-29 (2017).
104. N. Etemadi et al., TRAF2 regulates TNF and NF-κB signalling to suppress apoptosis and skin inflammation independently of Sphingosine kinase 1. Elife 4, (2015).
105. M. P. DeBerge, K. H. Ely, P. F. Wright, E. B. Thorp, R. I. Enelow, Shedding of TNF receptor 2 by effector CD8⁺ T cells by ADAM17 is important for regulating TNF-α availability during influenza infection. J Leukoc Biol 98, 423-434 (2015).
106. M. Leclerc et al., Control of GVHD by regulatory T cells depends on TNF produced by T cells and TNFR2 expressed by regulatory T cells. Blood 128, 1651-1659 (2016).
107. A. Subramanian et al., Gene set enrichment analysis: a knowledge-based approach for interpreting genome-wide expression profiles. Proc Natl Acad Sci USA 102, 15545-15550 (2005).
108. M. I. Love, W. Huber, S. Anders, Moderated estimation of fold change and dispersion for RNA-seq data with DESeq2. Genome Biol 15, 550 (2014).
109. R. J. Gorwitz et al., Changes in the prevalence of nasal colonization with Staphylococcus aureus in the United States, 2001-2004. J Infect Dis 197, 1226-1234 (2008).
110. K. B. Min, J. Y. Min, Nasal colonization with methicillin-resistant Staphylococcus aureus associated with elevated homocysteine levels in the general US adults. Medicine (Baltimore) 98, e15499 (2019).
111. A. L. Hersh, H. F. Chambers, J. H. Maselli, R. Gonzales, National trends in ambulatory visits and antibiotic prescribing for skin and soft-tissue infections. Arch Intern Med 168, 1585-1591 (2008).
112. S. Bangert, M. Levy, A. A. Hebert, Bacterial resistance and impetigo treatment trends: a review. Pediatr Dermatol 29, 243-248 (2012).
113. A. C. Bowen et al., The Global Epidemiology of Impetigo: A Systematic Review of the Population Prevalence of Impetigo and Pyoderma. PLoS One 10, e0136789 (2015).

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A method for treating a bacterial skin infection in a subject, the method comprising administering to a subject in need of treatment thereof a therapeutically effective amount of a tumor necrosis factor receptor 2 (TNFR2) agonist.

2. The method of claim 1, wherein the TNFR2 agonist is a monoclonal antibody (mAb).

3. The method of claim 2, wherein the mAb is selected from the group consisting of BI-1910, HFB200301, and MM-401.

4. The method of any one of claims 1-3, wherein the bacterial skin infection is caused by a Gram-positive skin pathogen.

5. The method of any one of claims 1-3, wherein the bacterial skin infection is caused by a Gram-negative skin pathogen.

6. The method of any one of claims 1-3, wherein the bacterial skin infection is caused by antibiotic-resistant bacteria.

7. The method of any one of claims 1-3, wherein the bacterial skin infection is caused by Staphylococcus aureus (S. aureus).

8. The method of claim 7, wherein the bacterial skin infection is caused by methicillin-resistant S. aureus.

9. The method of any one of claims 1-3, wherein the bacterial skin infection is caused by Pseudomonas aeruginosa (P. aeruginosa).

10. The method of any one of claims 1-9, wherein the method comprises:
(i) reducing a size of one or more skin lesions caused by a bacterial infection as compared to a size of the one or more skin lesions prior to treatment; and/or
(ii) reducing a bacterial burden in the subject as compared to a bacterial burden prior to treatment.

11. The method of any one of claims 1-9, wherein the method comprises:
(i) accelerating a rate of reduction in size of one or more skin lesions formed after a bacterial infection as compared to a rate of reduction in size of one or more skin lesions formed after a bacterial infection in a subject not receiving the TNFR2 agonist; and/or
(ii) accelerating a rate of bacterial clearance after a bacterial infection as compared to a rate of bacterial clearance after a bacterial infection in a subject not receiving the TNFR2 agonist.

12. The method of any one of claims 1-9, wherein administering the TNFR2 agonist:
(i) reduces a size of one or more skin lesions formed after a bacterial infection as compared to a size of one or more skin lesions formed after a bacterial infection in a subject not administered the TNFR2 agonist; and/or
(ii) reduces a bacterial burden in the subject receiving the TNFR2 agonist as compared to a bacterial burden in a subject not administered the TNFR2 agonist.

13. A method for treating an inflammatory disease associated with neutrophil extracellular trap (NET) formation, the method comprising administering to a subject in need of treatment thereof a therapeutically effective amount of a TNFR2 antagonist.

14. The method of claim 13, wherein the TNFR2 antagonist is selected from the group consisting of BI-1808, BITR2101, APX601, AN3025, SIM0235, LBL-019, and NBL-020.

15. The method of claim 13 or 14, wherein the inflammatory disease associated with NET formation is selected from psoriasis, rheumatoid arthritis, and neutrophilic asthma.
